(19) United States
(12) Patent Application Publication
(10) Pub. No.: US 2012/0064064 A1
(43) Pub. Date: Mar. 15, 2012

(54) ANTIGEN-BINDING PROTEINS

(76) Inventors: Thil Dinuk Batuwangala, Cambridgeshire (GB); Laurent Jespers, Cambridgeshire (GB); Michael Steward, Hertfordshire (GB)

(21) Appl. No.: 13/322,229

(22) PCT Filed: May 26, 2010
(86) PCT No.: PCT/EP10/57233

§ 371 Date: Nov. 23, 2011

Related U.S. Application Data
(60) Provisional application No. 61/181,897, filed on May 28, 2009.

(51) Int. Cl. A61K 39/395; C12N 5/10; C12N 1/21; C07K 16/24; C07K 16/00; C07K 16/22; C07H 21/04; C12P 21/06; C12N 1/19

(52) U.S. Cl. 424/130.1; 435/69.6; 435/325; 435/348; 435/358; 435/365; 435/366; 435/252.3; 435/252.31; 435/252.33; 435/252.35; 435/254.2; 530/389.1; 530/389.2; 536/23.53

(57) ABSTRACT

The present invention relates to antigen binding proteins comprising an immunoglobulin heavy chain and an immunoglobulin light chain, wherein the heavy chain comprises an epitope binding domain linked to the n-terminus of CH1-CH2-CH3, and the light chain comprises an epitope binding domain linked to the n-terminus of CL, wherein one or more epitope-binding domains are linked to the C-terminus of the immunoglobulin heavy chain, and/or one or more epitope-binding domains are linked to the C-terminus of the immunoglobulin light chain, methods for making such proteins, and uses thereof.

### BACKGROUND

### Summary of Invention

The present invention in particular relates to an antigen-binding protein comprising an immunoglobulin heavy chain and an immunoglobulin light chain, wherein the heavy chain comprises an epitope binding domain linked to the n-terminus of CH1-CH2-CH3, and the light chain comprises an epitope binding domain linked to the n-terminus of CL, wherein one or more epitope-binding domains are linked to the C-terminus of the immunoglobulin heavy chain, and/or one or more epitope-binding domains are linked to the C-terminus of the immunoglobulin light chain.

The invention also provides a polynucleotide sequence encoding a heavy chain of any of the antigen-binding proteins described herein, and a polynucleotide encoding a light chain of any of the antigen-binding proteins described herein. Such polynucleotides represent the coding sequence which corresponds to the equivalent polypeptide sequences, however it will be understood that such polynucleotide sequences could be cloned into an expression vector along with a start codon, an appropriate signal sequence and a stop codon.

The invention also provides a recombinant transformed or transfected host cell comprising one or more polynucleotides encoding a heavy chain and a light chain of any of the antigen-binding proteins described herein.

The invention further provides a method for the production of any of the antigen-binding proteins described herein which method comprises the step of culturing a host cell comprising a vector, encoding any of the antigen-binding proteins described herein, for example in a serum-free culture media.

The invention further provides a pharmaceutical composition comprising an antigen-binding protein as described herein a pharmaceutically acceptable carrier.

The invention further provides the use of such antigen-binding proteins or pharmaceutical compositions such antigen-binding proteins in the treatment of immune diseases for example auto-immune diseases, or cancer, or inflammatory diseases.

### DEFINITIONS

The term “antigen binding protein” as used herein refers to antibodies, antibody fragments and other protein constructs which are capable of binding to antigens.

The term ‘Antibody scaffold’ as used herein refers to a scaffold comprising an immunoglobulin heavy chain composed of CH1-CH2-CH3 and an immunoglobulin light chain comprising CL.

The term CH1 as used herein refers to the constant domain 1 of an immunoglobulin heavy chain.

The term CH2 as used herein refers to the constant domain 2 of an immunoglobulin heavy chain.

The term CH3 as used herein refers to the constant domain 3 of an immunoglobulin heavy chain.

The term CL as used herein refers to the constant domain of an immunoglobulin light chain.

A “domain” is a folded protein structure which has tertiary structure independent of the rest of the protein. Generally, domains are responsible for discrete functional properties of proteins and in many cases may be added, removed or transferred to other proteins without loss of function of the remainder of the protein and/or of the domain. A “single antibody variable domain” is a folded polypeptide domain comprising sequences characteristic of antibody variable domains. It therefore includes complete antibody variable domains and modified variable domains, for example, in which one or more loops have been replaced by sequences which are not characteristic of antibody variable domains, or antibody variable domains which have been truncated or comprise N- or C-terminal extensions, as well as folded fragments of variable domains which retain at least the binding activity and specificity of the full-length domain.

The phrase “immunoglobulin single variable domain” refers to an antibody variable domain (V_{H}, V_{HH}, V_{L}) that specifically binds an antigen or epitope independently of a different V region or domain. An immunoglobulin single variable domain can be present in a format (e.g., homo- or hetero-multimer) with other, different variable regions or variable domains where the other regions or domains are not required for antigen binding by the single immunoglobulin variable domain (i.e., where the immunoglobulin single variable domain binds antigen independently of the additional variable domains). A “domain antibody” or “dAb” is the same as an “immunoglobulin single variable domain” which is capable of binding to an antigen as the term is used herein. An immunoglobulin single variable domain may be a human antibody variable domain, but also includes single antibody variable domains from other species such as rodent (for example, as disclosed in WO 00/29004), nurse shark and Camelid V_{HH} immunoglobulin single variable domains. Camelid V_{HH} are immunoglobulin single variable domain polypeptides that are derived from species including camel, llama, alpaca, dromedary, and guanaco, which produce heavy chain antibodies naturally devoid of light chains. Such V_{HH} domains may be humanised according to standard techniques available in the art, and such domains are still considered to be “domain antibodies” according to the invention. As used herein “V_{H} includes camelid V_{HH} domains. NARV are another type of immunoglobulin single variable domain which were identified in cartilaginous fish including the nurse shark. These domains are also known as Novel Antigen Receptor variable region (commonly abbreviated to V(NAR) or NARV). For further details see Mol. Immunol. 44, 656-665 (2006) and US20050043519A.

The term “Epitope-binding domain” refers to a domain that specifically binds an antigen or epitope independently of a different V region or domain, this may be an immunoglobulin single variable domain, for example a human dAb (domain antibody), camelid or shark immunoglobulin single variable domain or it may be a non-Ig domain which has been subjected to protein engineering in order to obtain binding to a ligand other than its natural ligand, for example a domain which is a derivative of a scaffold selected from the group consisting of CTLA-4 (Evibody); lipocalin; Protein A derived molecules such as Z-domain of Protein A (Affibody, SpA), A-domain (Avimer/Maxibody); Heat shock proteins such as GroEI and GroES; transferrin (trans-body); ankyrin repeat protein (DARPin); peptide aptamer; C-type lectin domain (Tetranectin); human γ-crystallin and human ubiquitin (affilins); PDZ domains; scorpion toxinkunitz type domains of human protease inhibitors; and fibronectin (adnectin); which has been subjected to protein engineering in order to obtain binding to a ligand other than its natural ligand.

CTLA-4 (Cytotoxic T Lymphocyte-associated Antigen 4) is a CD28-family receptor expressed on mainly CD4+ T-cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies. For further details see Journal of Immunological Methods 248 (1-2), 31-45 (2001)

Lipocalins are a family of extracellular proteins which transport small hydrophobic molecules such as steroids, bilins, retinoids and lipids. They have a rigid β-sheet secondary structure with a numer of loops at the open end of the conical structure which can be engineered to bind to different target antigens. Anticalins are between 160-180 amino acids in size, and are derived from lipocalins. For further details see Biochim Biophys Acta 1482: 337-350 (2000), U.S. Pat. No. 7,250,297B1 and US20070224633

An affibody is a scaffold derived from Protein A of Staphylococcus aureus which can be engineered to bind to antigen. The domain consists of a three-helical bundle of approximately 58 amino acids. Libraries have been generated by randomisation of surface residues. For further details see Protein Eng. Des. Sel. 17, 455-462 (2004) and EP1641818A1

Avimers are multidomain proteins derived from the A-domain scaffold family. The native domains of approximately 35 amino acids adopt a defined disulphide bonded structure. Diversity is generated by shuffling of the natural variation exhibited by the family of A-domains. For further details see Nature Biotechnology 23(12), 1556-1561 (2005) and Expert Opinion on Investigational Drugs 16(6), 909-917 (June 2007)

A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem. 274, 24066-24073 (1999).

Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two α-helices and a β-turn. They can be engineered to bind different target antigens by randomising residues in the first α-helix and a β-turn of each repeat. Their binding interface can be increased by increasing the number of modules (a method of affinity maturation). For further details see J. Mol. Biol. 332, 489-503 (2003), PNAS 100(4), 1700-1705 (2003) and J. Mol. Biol. 369, 1015-1028 (2007) and US20040132028A1.

Fibronectin is a scaffold which can be engineered to bind to antigen. Adnectins consists of a backbone of the natural amino acid sequence of the 10th domain of the 15 repeating units of human fibronectin type III (FN3). Three loops at one end of the β-sandwich can be engineered to enable an Adnectin to specifically recognize a therapeutic target of interest. For further details see Protein Eng. Des. Sel. 18, 435-444 (2005), US20080139791, WO2005056764 and U.S. Pat. No. 6,818,418B1.

Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005).

Microbodies are derived from naturally occurring microproteins of 25-50 amino acids in length which contain 3-4 cysteine bridges—examples of microproteins include KalataB1 and conotoxin and knottins. The microproteins have a loop which can be engineered to include upto 25 amino acids without affecting the overall fold of the microprotein. For further details of engineered knottin domains, see WO2008098796.

Other epitope binding domains include proteins which have been used as a scaffold to engineer different target antigen binding properties include human γ-crystallin and human ubiquitin (affilins), kunitz type domains of human protease inhibitors, PDZ-domains of the Ras-binding protein AF-6, scorpion toxins (charybdotoxin), C-type lectin domain (tetranectins) are reviewed in Chapter 7—Non-Antibody Scaffolds from Handbook of Therapeutic Antibodies (2007, edited by Stefan Dubel) and Protein Science 15:14-27 (2006). Epitope binding domains of the present invention could be derived from any of these alternative protein domains.

In one embodiment of the invention the antigen-binding site binds to antigen with a Kd of at least 1 mM, for example a Kd of 10 nM, 1 nM, 500 pM, 200 pM, 100 pM, to each antigen as measured by Biacore™, such as the Biacore™ method as described in method 4 or 5.

As used herein, the term “antigen-binding site” refers to a site on a protein which is capable of specifically binding to antigen, this may be a single domain, for example an epitope-binding domain, or it may be the portion of the soluble ligand or extracellular domain of a receptor or cell surface protein which is capable of binding antigen.

### DETAILED DESCRIPTION OF INVENTION

The present invention provides an antigen-binding protein comprising an immunoglobulin heavy chain and an immunoglobulin light chain, wherein the heavy chain comprises an epitope binding domain linked to the n-terminus of CH1-CH2-CH3, and the light chain comprises an epitope binding domain linked to the n-terminus of CL, wherein one or more epitope-binding domains are linked to the C-terminus of the immunoglobulin heavy chain, and/or one or more epitope-binding domains are linked to the C-terminus of the immunoglobulin light chain.

In one embodiment the antigen binding protein of the invention will have two identical heavy chains and two identical light chains and these heavy and light chains when expressed in a cell will associate to form an antibody-like, hetero-tetrameric structure.

The antigen binding proteins of the present invention comprise an antibody scaffold.

This may be selected from antibodies of any isotype, for example IgG1, IgG2, IgG3, IgG4 or IgG4PE.

In one embodiment of the present invention at least one epitope binding domain is an immunoglobulin single variable domain. In another embodiment the epitope binding domains linked to the n-terminus of the heavy chain and the n-terminus of the light chain are immunoglobulin single variable domains. In yet another embodiment all the epitope binding domains are immunoglobulin single variable domains.

In one embodiment of the present invention at least one epitope binding domain is a non-Ig domain. In another embodiment the epitope binding domains linked to the n-terminus of the heavy chain and the n-terminus of the light chain are immunoglobulin single variable domains and the epitope binding domains linked to the c-terminus of the heavy chain and/or the c-terminus of the light chain are non-Ig domains. In another embodiment the epitope binding domains linked to the n-terminus of the heavy chain and the n-terminus of the light chain are non-Ig domains and the epitope binding domains linked to the c-terminus of the heavy chain and/or the c-terminus of the light chain are immunoglobulin single variable domains. In yet another embodiment all the epitope binding domains are non-Ig domains.

It will be understood that any of the antigen-binding proteins described herein will be capable of neutralising one or more antigens.

The term “neutralises” and grammatical variations thereof as used throughout the present specification in relation to antigen-binding proteins of the invention means that a biological activity of the target is reduced, either totally or partially, in the presence of the antigen-binding proteins of the present invention in comparison to the activity of the target in the absence of such antigen-binding proteins.

Neutralisation may be due to but not limited to one or more of blocking ligand binding, preventing the ligand activating the receptor, down regulating the receptor or affecting effector functionality.

Levels of neutralisation can be measured in several ways, for example by use of any of the assays as set out in the examples below, for example in an assay which measures inhibition of ligand binding to receptor which may be carried out for example as described in Example 3. The neutralisation of VEGFR2, in this assay is measured by assessing the decreased binding between the ligand and its receptor in the presence of neutralising antigen-binding protein.

Other methods of assessing neutralisation, for example, by assessing the decreased binding between the ligand and its receptor in the presence of neutralising antigen-binding protein are known in the art, and include, for example, Biacore™ assays.

In an alternative aspect of the present invention there is provided antigen-binding proteins which have at least substantially equivalent neutralising activity to the antigen-binding proteins exemplified herein.

In one embodiment the antigen-binding proteins of the invention have specificity for VEGF, for example they comprise at least one epitope binding domain which binds to VEGF, for example an immunoglobulin single variable domain, an anticalin, or an adnectin which binds to VEGF.

In one embodiment the antigen-binding proteins of the invention have specificity for VEGFR2, for example they comprise at least one epitope binding domain which binds to VEGFR2, for example an immunoglobulin single variable domain or an adnectin which binds to VEGFR2.

In one embodiment the antigen-binding proteins of the invention have specificity for TNFα, for example they comprise at least one epitope binding domain which binds to TNFα, for example an immunoglobulin single variable domain or an adnectin which binds to TNFα.

In one embodiment the antigen-binding proteins of the invention have specificity for IL-13, for example they comprise at least one epitope binding domain which binds to IL-13, for example an immunoglobulin single variable domain or an adnectin which binds to IL-13.

In one embodiment the antigen-binding proteins of the invention have specificity for HER2, for example they comprise at least one epitope binding domain which binds to HER2, for example an immunoglobulin single variable domain or an adnectin which binds to HER2.

In one embodiment the antigen-binding protein of the present invention has specificity for only one antigen, for example, the present invention provides an antigen binding protein capable of binding TNFα.

In an alternative embodiment the antigen-binding protein of the present invention has specificity for more than one antigen, for example, the present invention provides an antigen binding protein capable of binding to two or more antigens selected from, VEGF, TNFα and EGFR for example, an antigen binding protein capable of binding VEGF and EGFR, or an antigen binding protein capable of binding VEGF and TNFα, or an antigen binding protein capable of binding TNFα and EGFR.

In a further embodiment the antigen-binding protein of the present invention has specificity for at least three antigens, for example, the present invention provides a an antigen binding protein capable of binding to, VEGF, TNFα and EGFR for example, an antigen binding protein comprising the epitope binding domains set out in SEQ ID NO: 7, 8 and 9.

In one embodiment the antigen-binding protein of the present invention is capable of binding VEGF and EGFR simultaneously, or TNFα and VEGF simultaneously, or TNFα and EGFR or TNFα and VEGF and EGFR simultaneously.

It will be understood that any of the antigen-binding proteins described herein may be capable of binding two or more antigens simultaneously, for example, as determined by stochiometry analysis by using a suitable assay such as that described in Example 6.

Examples of such antigen-binding proteins include antigen binding proteins comprising an epitope binding domain with a specificity for VEGF, for example an anti-VEGF immunoglobulin single variable domain or anti-VEGF anticalin, for example the dAb set out in SEQ ID NO: 7, or the anticalin set out in SEQ ID NO: 41

Examples of such antigen-binding proteins include an antigen binding proteins comprising an epitope binding domain with a specificity for VEGFR2, for example the adnectin set out in SEQ ID NO: 39.

Examples of such antigen-binding proteins include antigen binding proteins comprising an epitope binding domain with a specificity for EGFR, for example an anti-EGFR immunoglobulin single variable domain, for example the dAb set out in SEQ ID NO: 8.

Examples of such antigen-binding proteins include antigen binding proteins comprising an epitope binding domain with a specificity for EGFR, for example an anti-II1R1 immunoglobulin single variable domain, for example the dAb set out in SEQ ID NO: 9.

Examples of such antigen-binding proteins include antigen binding proteins comprising an epitope binding domain with a specificity for TNFα, for example an anti-TNFα adnectin, for example the adnectin set out in SEQ ID NO:40.

The epitope binding domains with the same or different antigen-specificity attached to its c-terminus or the n-terminus.

In one embodiment of the present invention there is provided an antigen binding protein according to the invention described herein and comprising a constant region such that the antigen binding protein has reduced ADCC and/or complement activation or effector functionality. In one such embodiment the heavy chain constant region may comprise a naturally disabled constant region of IgG2 or IgG4 isotype or a mutated IgG1 constant region. Examples of suitable modifications are described in EP0307434. One example comprises the substitutions of alanine residues at positions 235 and 237 (EU index numbering).

In one embodiment the antigen-binding proteins of the present invention will retain Fc functionality for example will be capable of one or both of ADCC and CDC activity.

The antigen-binding proteins of the invention may have some effector function. For example if the antibody scaffold contains an Fc region derived from an antibody with effector function, for example if the antibody scaffold comprises CH2 and CH3 from IgG1. Levels of effector function can be varied according to known techniques, for example by mutations in the CH2 domain, for example wherein the IgG1 CH2 domain has one or more mutations at positions selected from 239 and 332 and 330, for example the mutations are selected from S239D and I332E and A330L such that the antibody has enhanced effector function, and/or for example altering the glycosylation profile of the antigen-binding protein of the invention such that there is a reduction in fucosylation of the Fc region.

In one embodiment, the antigen-binding proteins comprise an epitope-binding domain which is an immunoglobulin single variable domain, for example the epitope binding domain may be a human VH or human VL, or a camelid V_{HH} or a shark immunoglobulin single variable domain (NARV).

In one embodiment the antigen-binding proteins comprise an epitope-binding domain which is a derivative of a non-Ig scaffold selected from the group consisting of CTLA-4 (Evibody); lipocalin; Protein A derived molecules such as Z-domain of Protein A (Affibody, SpA), A-domain (Avimer/Maxibody); Heat shock proteins such as GroEI and GroES; transferrin (trans-body); ankyrin repeat protein (DARPin); peptide aptamer; C-type lectin domain (Tetranectin); human γ-crystallin and human ubiquitin (affilins); PDZ domains; scorpion toxinkunitz type domains of human protease inhibitors; and fibronectin (adnectin); which has been subjected to protein engineering in order to obtain binding to a ligand other than its natural ligand.

In one embodiment of the present invention there are six epitope binding domains, for example, one epitope binding domain linked to the n-terminus of each heavy chain of the antibody scaffold, one epitope binding domain linked to the n-terminus of each light chain of the antibody scaffold and one epitope binding domain linked to the c-terminus of each heavy chain of the antibody scaffold, or for example one epitope binding domain linked to the n-terminus of each heavy chain of the antibody scaffold, one epitope binding domain linked to the n-terminus of each light chain of the antibody scaffold and one epitope binding domain linked to the c-terminus of each light chain of the antibody scaffold.

In another embodiment of the present invention there are eight epitope binding domains, for example, one epitope binding domain linked to the n-terminus of each heavy chain of the antibody scaffold, one epitope binding domain linked to the n-terminus of each light chain of the antibody scaffold, one epitope binding domain linked to the c-terminus of each heavy chain of the antibody scaffold and one epitope binding domain linked to the c-terminus of each light chain of the antibody scaffold.

Two of the epitope binding domains may have specificity for the same antigen, or four, or all of the epitope binding domains present in the antigen-binding protein may have specificity for the same antigen.

Antibody scaffolds of the present invention may be linked to epitope-binding domains by the use of linkers. Examples of suitable linkers include amino acid sequences which may be from 1 amino acid to 150 amino acids in length, or from 1 amino acid to 140 amino acids, for example, from 1 amino acid to 130 amino acids, or from 1 to 120 amino acids, or from 1 to 80 amino acids, or from 1 to 50 amino acids, or from 1 to 20 amino acids, or from 1 to 10 amino acids, or from 5 to 18 amino acids. Such sequences may have their own tertiary structure, for example, a linker of the present invention may comprise a single variable domain. The size of a linker in one embodiment is equivalent to a single variable domain. Suitable linkers may be of a size from 1 to 20 angstroms, for example less than 15 angstroms, or less than 10 angstroms, or less than 5 angstroms.

In one embodiment of the present invention at least one of the epitope binding domains is directly attached to the antibody scaffold with a linker comprising from 1 to 150 amino acids, for example 1 to 50 amino acids, for example 1 to 20 amino acids, for example 1 to 10 amino acids. Such linkers may be selected from any one of those set out in SEQ ID NO: 10-38 or SEQ ID NO:42-44, or multiples of such linkers.

Linkers of use in the antigen-binding proteins of the present invention may comprise alone or in addition to other linkers, one or more sets of GS residues, for example ‘GSTVAAPS’ (SEQ ID NO:44) or ‘TVAAPSGS’ (SEQ ID NO: 11) or ‘GSTVAAPSGS’ (SEQ ID NO:18).

In one embodiment the epitope binding domain is linked to the antibody scaffold by the linker ‘(PAS)ₙ(GS)ₘ’. In another embodiment the epitope binding domain is linked to the antibody scaffold by the linker ‘(GGGGS)ₙ(GS)ₘ’. In another embodiment the epitope binding domain is linked to the antibody scaffold by the linker ‘(TVAAPS)ₙ(GS)ₘ’. In another embodiment the epitope binding domain is linked to the antibody scaffold by the linker ‘(GS)ₘ(TVAAPSGS)ₙ’. In another embodiment the epitope binding domain is linked to the antibody scaffold by the linker ‘(PAVPPP)ₙ(GS)ₘ’. In another embodiment the epitope binding domain is linked to the antibody scaffold by the linker ‘(TVSDVP)ₙ(GS)ₘ’. In another embodiment the epitope binding domain is linked to the antibody scaffold by the linker ‘(TGLDSP)ₙ(GS)ₘ’. In all such embodiments, n=1-10, and m=0-4.

Examples of such linkers include (PAS)ₙ(GS)ₘ wherein n=1 and m=1 (SEQ ID NO:24), (PAS)ₙ(GS)ₘ wherein n=2 and m=1 (SEQ ID NO:25), (PAS)ₙ(GS)ₘ wherein n=3 and m=1 (SEQ ID NO:26), (PAS)ₙ(GS)ₘ wherein n=4 and m=1, (PAS)ₙ(GS)ₘ wherein n=2 and m=0, (PAS)ₙ(GS)ₘ wherein n=3 and m=0, (PAS)ₙ(GS)ₘ wherein n=4 and m=0.

Examples of such linkers include (GGGGS)ₙ(GS)ₘ wherein n=1 and m=1, (GGGGS)ₙ(GS)ₘ wherein n=2 and m=1, (GGGGS)ₙ(GS)ₘ wherein n=3 and m=1, (GGGGS)ₙ(GS)ₘ wherein n=4 and m=1, (GGGGS)ₙ(GS)ₘ wherein n=2 and m=0 (SEQ ID NO:28), (GGGGS)ₙ(GS)ₘ wherein n=3 and m=0 (SEQ ID NO:29), (GGGGS)ₙ(GS)ₘ wherein n=4 and m=0.

Examples of such linkers include (TVAAPS)ₙ(GS)ₘ wherein n=1 and m=1, (TVAAPS)ₙ(GS)ₘ wherein n=2 and m=1, (TVAAPS)ₙ(GS)ₘ wherein n=3 and m=1, (TVAAPS)ₙ(GS)ₘ wherein n=4 and m=1, (TVAAPS)ₙ(GS)ₘ wherein n=2 and m=0, (TVAAPS)ₙ(GS)ₘ wherein n=3 and m=0, (TVAAPS)ₙ(GS)ₘ wherein n=4 and m=0.

Examples of such linkers include (GS)ₘ(TVAAPSGS)ₙ wherein n=1 and m=1 (SEQ ID NO:18), (GS)ₘ(TVAAPSGS)ₙ wherein n=2 and m=1 (SEQ ID NO:19), (GS)ₘ(TVAAPSGS)ₙ wherein n=3 and m=1 (SEQ ID NO:20), or (GS)ₘ(TVAAPSGS)ₙ wherein n=4 and m=1 (SEQ ID NO:21), (GS)ₘ(TVAAPSGS)ₙ wherein n=5 and m=1 (SEQ ID NO:22), (GS)ₘ(TVAAPSGS)ₙ wherein n=6 and m=1 (SEQ ID NO:23), (GS)ₘ(TVAAPSGS)ₙ wherein n=1 and m=0 (SEQ ID NO:11), (GS)ₘ(TVAAPSGS)ₙ wherein n=2 and m=10, (GS)ₘ(TVAAPSGS)ₙ wherein n=3 and m=0, or (GS)ₘ(TVAAPSGS)ₙ wherein n=0.

Examples of such linkers include (PAVPPP)ₙ(GS)ₘ wherein n=1 and m=1 (SEQ ID NO:14), (PAVPPP)ₙ(GS)ₘ wherein n=2 and m=1, (PAVPPP)ₙ(GS)ₘ wherein n=3 and m=1, (PAVPPP)ₙ(GS)ₘ wherein n=4 and m=1, (PAVPPP)ₙ(GS)ₘ wherein n=2 and m=0, (PAVPPP)ₙ(GS)ₘ wherein n=3 and m=0, (PAVPPP)ₙ(GS)ₘ wherein n=4 and m=0.

Examples of such linkers include (TVSDVP)ₙ(GS)ₘ wherein n=1 and m=1 (SEQ ID NO:15), (TVSDVP)ₙ(GS)ₘ wherein n=2 and m=1, (TVSDVP)ₙ(GS)ₘ wherein n=3 and m=1, (TVSDVP)ₙ(GS)ₘ wherein n=4 and m=1, (TVSDVP)ₙ(GS)ₘ wherein n=2 and m=0, (TVSDVP)ₙ(GS)ₘ wherein n=3 and m=0, (TVSDVP)ₙ(GS)ₘ wherein n=4 and m=0.

Examples of such linkers include (TGLDSP)ₙ(GS)ₘ wherein n=1 and m=1 (SEQ ID NO:16), (TGLDSP)ₙ(GS)ₘ wherein n=2 and m=1, (TGLDSP)ₙ(GS)ₘ wherein n=3 and m=1, (TGLDSP)ₙ(GS)ₘ wherein n=4 and m=1, (TGLDSP)ₙ(GS)ₘ wherein n=2 and m=0, (TGLDSP)ₙ(GS)ₘ wherein n=3 and m=0, (TGLDSP)ₙ(GS)ₘ wherein n=4 and m=0.

In another embodiment there is no linker between the epitope binding domain and the antibody scaffold. In another embodiment the epitope binding domain is linked to the antibody scaffold by the linker ‘TVAAPS’. In another embodiment the epitope binding domain, is linked to the antibody scaffold by the linker ‘TVAAPSGS’. In another embodiment the epitope binding domain is linked to the antibody scaffold by the linker ‘GS’. In another embodiment the epitope binding domain is linked to the antibody scaffold by the linker ‘ASTKGPT’.

In one embodiment, the antigen-binding protein of the present invention comprises at least one epitope binding domain, which is capable of binding human serum albumin.

The invention also provides the antigen-binding proteins for use in medicine. The antigen binding proteins may be useful in treating diseases which are susceptible to inhibition of angiogenic pathways and blocking of pro-inflammatory cytokines, for example for use in the manufacture of a medicament for treating immune diseases for example auto-immune diseases, or cancer, or inflammatory diseases, for example systemic lupus erythramatosis, multiple sclerosis, crohns disease, psoriasis, or arthritic diseases, for example rheumatoid arthritis.

The invention provides a method of treating a patient suffering from immune diseases for example auto-immune diseases, or cancer, or inflammatory diseases, for example systemic lupus erythramatosis, multiple sclerosis, crohns disease, psoriasis, or arthritic diseases, for example rheumatoid arthritis, comprising administering a therapeutic amount of an antigen-binding protein of the invention.

The antigen-binding proteins of the invention may be used for the treatment of immune diseases for example auto-immune diseases, or cancer, or inflammatory diseases, for example systemic lupus erythramatosis, multiple sclerosis, crohns disease, psoriasis, or arthritic diseases, for example rheumatoid arthritis.

Epitope-binding domains of use in the present invention are domains that specifically bind an antigen or epitope independently of a different V region or domain, this may be a domain antibody or may be a domain which is a derivative of a scaffold selected from the group consisting of CTLA-4 (Evibody); lipocalin; Protein A derived molecules such as Z-domain of Protein A (Affibody, SpA), A-domain (Avimer/Maxibody); Heat shock proteins such as GroEI and GroES; transferrin (trans-body); ankyrin repeat protein (DARPin); peptide aptamer; C-type lectin domain (Tetranectin); human γ-crystallin and human ubiquitin (affilins); PDZ domains; scorpion toxinkunitz type domains of human protease inhibitors; and fibronectin (adnectin); which has been subjected to protein engineering in order to obtain binding to a ligand other than its natural ligand. In one embodiment this may be a domain antibody or other suitable domains such as a domain selected from the group consisting of CTLA-4, lipocallin, SpA, an Affibody, an avimer, GroEI, transferrin, GroES and fibronectin. In one embodiment this may be selected from an immunoglobulin single variable domain, an Affibody, an ankyrin repeat protein (DARPin) and an adnectin. In another embodiment this may be selected from an Affibody, an ankyrin repeat protein (DARPin) and an adnectin. In another embodiment this may be a domain antibody, for example a domain antibody selected from a human, camelid or shark (NARV) domain antibody.

Epitope-binding domains can be linked to the antibody scaffold at one or more positions. These positions include the C-terminus and the N-terminus of the antibody scaffold. For example they may be linked directly to the Fc portion of the antibody scaffold, or they may be linked to the soluble ligand or extracellular domain of a receptor or cell surface protein portion of the antibody scaffold. Where the soluble ligand or extracellular domain of a receptor or cell surface protein is linked to the N-terminus of the Fc portion, the epitope-binding domain may be linked directly to the c-terminus of the Fc portion or to the N-terminus of the soluble ligand or extracellular domain of a receptor or cell surface protein.

In one embodiment, a first epitope binding domain is linked to the antibody scaffold and a second epitope binding domain is linked to the first epitope binding domain, for example a first epitope binding domain may be linked to the c-terminus of the antibody scaffold, and that epitope binding domain can be linked at its c-terminus to a second epitope binding domain, or for example a first epitope binding domain may be linked to the n-terminus of the antibody scaffold, and that first epitope binding domain may be further linked at its n-terminus to a second epitope binding domain, When the epitope-binding domain is a domain antibody, some domain antibodies may be suited to particular positions within the scaffold.

In antigen binding proteins where the N-terminus of ns are fused to an antibody constant domain, a peptide linker between the immunoglobulin single variable domain and the Fc portion may help the immunoglobulin single variable domain to bind to antigen. Indeed, the N-terminal end of an immunoglobulin single variable domain is located closely to the complementarity-determining regions (CDRs) involved in antigen-binding activity. Thus a short peptide linker acts as a spacer between the epitope-binding, and the Fc portion, which may allow the immunoglobulin single variable domain CDRs to more easily reach the antigen, which may therefore bind with high affinity.

The surroundings in which immunoglobulin single variable domains are linked to the IgG will differ depending on which antibody chain they are fused to:

When fused at the C-terminal end of the Fc portion, each immunoglobulin single variable domain is expected to be located in the vicinity of the C_{H}3 domains of the Fc portion. This is not expected to impact on the Fc binding properties to Fc receptors (e.g. FcγRI, II, III an FcRn) as these receptors engage with the C_{H}2 domains (for the FcγRI, II and III class of receptors) or with the hinge between the C_{H}2 and C_{H}3 domains (e.g. FcRn receptor). Another feature of such antigen-binding proteins is that both immunoglobulin single variable domains are expected to be spatially close to each other and provided that flexibility is provided by provision of appropriate linkers, these immunoglobulin single variable domains may even form homodimeric species, hence propagating the ‘zipped’ quaternary structure of the Fc portion, which may enhance stability of the protein.

Such structural considerations can aid in the choice of the most suitable position to link an epitope-binding domain, for example an immunoglobulin single variable domain, on to an antibody scaffold.

Understanding the solution state and mode of binding at the immunoglobulin single variable domain is also helpful. Evidence has accumulated that in vitro dAbs can predominantly exist in monomeric, homo-dimeric or multimeric forms in solution (Reiter et al. (1999) J Mol Biol 290 p 685-698; Ewert et al (2003) J Mol Biol 325, p 531-553, Jespers et al (2004) J Mol Biol 337 p 893-903; Jespers et al (2004) Nat Biotechnol 22 p 1161-1165; Martin et al (1997) Protein Eng. 10 p 607-614; Sepulvada et al (2003) J Mol Biol 333 p 355-365). This is fairly reminiscent to multimerisation events observed in vivo with Ig domains such as Bence-Jones proteins (which are dimers of immunoglobulin light chains (Epp et al (1975) Biochemistry 14 p 4943-4952; Huan et al (1994) Biochemistry 33 p 14848-14857; Huang et al (1997) Mol immunol 34 p 1291-1301) and amyloid fibers (James et al. (2007) J Mol. Biol. 367:603-8).

For example, it may be desirable to link domain antibodies that tend to dimerise in solution to the C-terminal end of the Fc portion in preference to the N-terminal end of the antibody scaffold as linking to the C-terminal end of the Fc will allow those immunoglobulin single variable domains to dimerise more easily in the context of the antigen-binding protein of the invention.

The antigen-binding proteins of the present invention may comprise antigen-binding sites specific for a single antigen, or may have antigen-binding sites specific for two or more antigens, or for two or more epitopes on a single antigen, or there may be antigen-binding sites each of which is specific for a different epitope on the same or different antigens.

The invention also provides the antigen-binding proteins for use in medicine, for example for use in the manufacture of a medicament for treating immune diseases for example auto-immune diseases, or cancer, or inflammatory diseases, for example systemic lupus erythramatosis, multiple sclerosis, crohns disease, psoriasis, or arthritic diseases, for example rheumatoid arthritis.

In particular, the antigen-binding proteins of the present invention may be useful in treating immune diseases for example auto-immune diseases, or cancer, or inflammatory diseases, for example systemic lupus erythramatosis, multiple sclerosis, crohns disease, psoriasis, or arthritic diseases, for example rheumatoid arthritis.

The invention provides a method of treating a patient suffering from immune diseases for example auto-immune diseases, or cancer, or inflammatory diseases, for example systemic lupus erythramatosis, multiple sclerosis, crohns disease, psoriasis, or arthritic diseases, for example rheumatoid arthritis comprising administering a therapeutic amount of an antigen-binding protein of the invention.

The antigen-binding proteins of the present invention may be produced by transfection of a host cell with an expression vector comprising the coding sequence for the antigen-binding protein of the invention. An expression vector or recombinant plasmid is produced by placing these coding sequences for the antigen-binding protein in operative association with conventional regulatory control sequences capable of controlling the replication and expression in, and/or secretion from, a host cell. Regulatory sequences include promoter sequences, e.g., CMV promoter, and signal sequences which can be derived from other known antibodies.

A selected host cell is transfected by conventional techniques with the vector to create the transfected host cell of the invention comprising the recombinant or synthetic heavy chains. The transfected cell is then cultured by conventional techniques to produce the engineered antigen-binding protein of the invention. The antigen-binding protein is screened from culture by appropriate assay, such as ELISA or RIA. Similar conventional techniques may be employed to construct other antigen-binding proteins.

Suitable vectors for the cloning and subcloning steps employed in the methods and construction of the compositions of this invention may be selected by one of skill in the art. For example, the conventional pUC series of cloning vectors may be used. One vector, pUC19, is commercially available from supply houses, such as Amersham (Buckinghamshire, United Kingdom) or Pharmacia (Uppsala, Sweden). Additionally, any vector which is capable of replicating readily, has an abundance of cloning sites and selectable genes (e.g., antibiotic resistance), and is easily manipulated may be used for cloning. Thus, the selection of the cloning vector is not a limiting factor in this invention.

The expression vectors may also be characterized by genes suitable for amplifying expression of the heterologous DNA sequences, e.g., the mammalian dihydrofolate reductase gene (DHFR). Other vector sequences include a poly A signal sequence, such as from bovine growth hormone (BGH) and the betaglobin promoter sequence (betaglopro). The expression vectors useful herein may be synthesized by techniques well known to those skilled in this art.

The components of such vectors, e.g. replicons, selection genes, enhancers, promoters, signal sequences and the like, may be obtained from commercial or natural sources or synthesized by known procedures for use in directing the expression and/or secretion of the product of the recombinant DNA in a selected host. Other appropriate expression vectors of which numerous types are known in the art for mammalian, bacterial, insect, yeast, and fungal expression may also be selected for this purpose.

The present invention also encompasses a cell line transfected with a recombinant plasmid containing the coding sequences of the antigen-binding proteins of the present invention. Host cells useful for the cloning and other manipulations of these cloning vectors are also conventional. However, cells from various strains of E. coli may be used for replication of the cloning vectors and other steps in the construction of antigen-binding proteins of this invention. Suitable host cells or cell lines for the expression of the antigen-binding proteins of the invention include mammalian cells such as NSO, Sp2/0, CHO (e.g. DG44), COS, HEK, a fibroblast cell (e.g., 3T3), and myeloma cells, for example it may be expressed in a CHO or a myeloma cell. Human cells may be used, thus enabling the molecule to be modified with human glycosylation patterns. Alternatively, other eukaryotic cell lines may be employed. The selection of suitable mammalian host cells and methods for transformation, culture, amplification, screening and product production and purification are known in the art. See, e.g., Sambrook et al., cited above.

Bacterial cells may prove useful as host cells suitable for the expression of the recombinant Fabs or other embodiments of the present invention (see, e.g., Plückthun, A., Immunol. Rev., 130:151-188 (1992)). However, due to the tendency of proteins expressed in bacterial cells to be in an unfolded or improperly folded form or in a non-glycosylated form, any recombinant antigen binding protein produced in a bacterial cell would have to be screened for retention of antigen binding ability. If the molecule expressed by the bacterial cell was produced in a properly folded form, that bacterial cell would be a desirable host, or in alternative embodiments the molecule may express in the bacterial host and then be subsequently re-folded. For example, various strains of E. coli used for expression are well-known as host cells in the field of biotechnology. Various strains of B. subtilis, Streptomyces, other bacilli and the like may also be employed in this method.

Where desired, strains of yeast cells known to those skilled in the art are also available as host cells, as well as insect cells, e.g. Drosophila and Lepidoptera and viral expression systems. See, e.g. Miller et al., Genetic Engineering, 8:277-298, Plenum Press (1986) and references cited therein.

The general methods by which the vectors may be constructed, the transfection methods required to produce the host cells of the invention, and culture methods necessary to produce the antigen-binding protein of the invention from such host cell may all be conventional techniques. Typically, the culture method of the present invention is a serum-free culture method, usually by culturing cells serum-free in suspension. Likewise, once produced, the antigen-binding proteins of the invention may be purified from the cell culture contents according to standard procedures of the art, including ammonium sulfate precipitation, affinity columns, column chromatography, gel electrophoresis and the like. Such techniques are within the skill of the art and do not limit this invention. For example, preparation of altered antibodies are described in WO 99/58679 and WO 96/16990.

Yet another method of expression of the antigen-binding proteins may utilize expression in a transgenic animal, such as described in U.S. Pat. No. 4,873,316. This relates to an expression system using the animal's casein promoter which when transgenically incorporated into a mammal permits the female to produce the desired recombinant protein in its milk.

In a further aspect of the invention there is provided a method of producing an antigen binding protein of the invention which method comprises the step of culturing a host cell transformed or transfected with a vector comprising a polynucleotide encoding the antigen binding protein of the invention and recovering the antigen binding protein thereby produced.

In accordance with the present invention there is provided a method of producing an antigen-binding protein of the present invention which method comprises the steps of;
(a) providing a vector comprising a polynucleotide encoding the antigen-binding protein
(b) transforming a mammalian host cell (e.g. CHO) with said vector;
(c) culturing the host cell of step (c) under conditions conducive to the secretion of the antigen-binding protein from said host cell into said culture media;
(d) recovering the secreted antigen-binding protein of step (d).

Once expressed by the desired method, the antigen-binding protein is then examined for in vitro activity by use of an appropriate assay. Presently conventional ELISA assay formats are employed to assess qualitative and quantitative binding of the antigen-binding protein to its target. Additionally, other in vitro assays may also be used to verify neutralizing efficacy prior to subsequent human clinical studies performed to evaluate the persistence of the antigen-binding protein in the body despite the usual clearance mechanisms.

The dose and duration of treatment relates to the relative duration of the molecules of the present invention in the human circulation, and can be adjusted by one of skill in the art depending upon the condition being treated and the general health of the patient. It is envisaged that repeated dosing (e.g. once a week or once every two weeks) over an extended time period (e.g. four to six months) maybe required to achieve maximal therapeutic efficacy.

The mode of administration of the therapeutic agent of the invention may be any suitable route which delivers the agent to the host. The antigen-binding proteins, and pharmaceutical compositions of the invention are particularly useful for parenteral administration, i.e., subcutaneously (s.c.), intrathecally, intraperitoneally, intramuscularly (i.m.), intravenously (i.v.), or intranasally.

Therapeutic agents of the invention may be prepared as pharmaceutical compositions containing an effective amount of the antigen-binding protein of the invention as an active ingredient in a pharmaceutically acceptable carrier. In the prophylactic agent of the invention, an aqueous suspension or solution containing the antigen-binding protein, may be buffered at physiological pH, in a form ready for injection. The compositions for parenteral administration will commonly comprise a solution of the antigen-binding protein of the invention or a cocktail thereof dissolved in a pharmaceutically acceptable carrier, for example an aqueous carrier. A variety of aqueous carriers may be employed, e.g., 0.9% saline, 0.3% glycine, and the like. These solutions may be made sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, etc. The concentration of the antigen-binding protein of the invention in such pharmaceutical formulation can vary widely, i.e., from less than about 0.5%, usually at or at least about 1% to as much as 15 or 20% by weight and will be selected primarily based on fluid volumes, viscosities, etc., according to the particular mode of administration selected.

Thus, a pharmaceutical composition of the invention for intramuscular injection could be prepared to contain 1 mL sterile buffered water, and between about 1 ng to about 100 mg, e.g. about 50 ng to about 30 mg, or about 5 mg to about 25 mg, of an antigen-binding protein of the invention. Similarly, a pharmaceutical composition of the invention for intravenous infusion could be made up to contain about 250 ml of sterile Ringer's solution, and about 1 to about 30 or about 5 mg to about 25 mg of an antigen-binding protein of the invention per ml of Ringer's solution. Actual methods for preparing parenterally administrable compositions are well known or will be apparent to those skilled in the art and are described in more detail in, for example, Remington's Pharmaceutical Science, 15th ed., Mack Publishing Company, Easton, Pa. For the preparation of intravenously administrable antigen-binding protein formulations of the invention see Lasmar U and Parkins D “The formulation of Biopharmaceutical products”, Pharma. Sci. Tech. today, page 129-137, Vol. 3 (3 Apr. 2000), Wang, W “Instability, stabilisation and formulation of liquid protein pharmaceuticals”, Int. J. Pharm 185 (1999) 129-188, Stability of Protein Pharmaceuticals Part A and B ed Ahern T. J., Manning M. C., New York, N.Y.: Plenum Press (1992), Akers, M. J. “Excipient-Drug interactions in Parenteral Formulations”, J. Pharm Sci 91 (2002) 2283-2300, Imamura, K et al “Effects of types of sugar on stabilization of Protein in the dried state”, J Pharm Sci 92 (2003) 266-274, Izutsu, Kkojima, S. “Excipient crystallinity and its protein-structure-stabilizing effect during freeze-drying”, J Pharm. Pharmacol, 54 (2002) 1033-1039, Johnson, R, “Mannitol-sucrose mixtures-versatile formulations for protein lyophilization”, J. Pharm. Sci, 91 (2002) 914-922.

Ha, E Wang W, Wang Y. j. “Peroxide formation in polysorbate 80 and protein stability”, J. Pharm Sci, 91, 2252-2264, (2002) the entire contents of which are incorporated herein by reference and to which the reader is specifically referred.

In one embodiment the therapeutic agent of the invention, when in a pharmaceutical preparation, is present in unit dose forms. The appropriate therapeutically effective dose will be determined readily by those of skill in the art. Suitable doses may be calculated for patients according to their weight, for example suitable doses may be in the range of 0.01 to 20 mg/kg, for example 0.1 to 20 mg/kg, for example 1 to 20 mg/kg, for example 10 to 20 mg/kg or for example 1 to 15 mg/kg, for example 10 to 15 mg/kg. To effectively treat conditions of use in the present invention in a human, suitable doses may be within the range of 0.01 to 1000 mg, for example 0.1 to 1000 mg, for example 0.1 to 500 mg, for example 500 mg, for example 0.1 to 100 mg, or 0.1 to 80 mg, or 0.1 to 60 mg, or 0.1 to 40 mg, or for example 1 to 100 mg, or 1 to 50 mg, of an antigen-binding protein of this invention, which may be administered parenterally, for example subcutaneously, intravenously or intramuscularly. Such dose may, if necessary, be repeated at appropriate time intervals selected as appropriate by a physician.

The antigen-binding proteins described herein can be lyophilized for storage and reconstituted in a suitable carrier prior to use. This technique has been shown to be effective with conventional immunoglobulins and art-known lyophilization and reconstitution techniques can be employed.

There are several methods known in the art which can be used to find epitope-binding domains of use in the present invention.

The term “library” refers to a mixture of heterogeneous polypeptides or nucleic acids. The library is composed of members, each of which has a single polypeptide or nucleic acid sequence. To this extent, “library” is synonymous with “repertoire.” Sequence differences between library members are responsible for the diversity present in the library. The library may take the form of a simple mixture of polypeptides or nucleic acids, or may be in the form of organisms or cells, for example bacteria, viruses, animal or plant cells and the like, transformed with a library of nucleic acids. In one example, each individual organism or cell contains only one or a limited number of library members. Advantageously, the nucleic acids are incorporated into expression vectors, in order to allow expression of the polypeptides encoded by the nucleic acids. In a one aspect, therefore, a library may take the form of a population of host organisms, each organism containing one or more copies of an expression vector containing a single member of the library in nucleic acid form which can be expressed to produce its corresponding polypeptide member. Thus, the population of host organisms has the potential to encode a large repertoire of diverse polypeptides.

A “universal framework” is a single antibody framework sequence corresponding to the regions of an antibody conserved in sequence as defined by Kabat (“Sequences of Proteins of Immunological Interest”, US Department of Health and Human Services) or corresponding to the human germline immunoglobulin repertoire or structure as defined by Chothia and Lesk, (1987) J. Mol. Biol. 196:910-917. There may be a single framework, or a set of such frameworks, which has been found to permit the derivation of virtually any binding specificity though variation in the hypervariable regions alone.

Amino acid and nucleotide sequence alignments and homology, similarity or identity, as defined herein are in one embodiment prepared and determined using the algorithm BLAST 2 Sequences, using default parameters (Tatusova, T. A. et al., FEMS Microbiol Lett, 174:187-188 (1999)).

When a display system (e.g., a display system that links coding function of a nucleic acid and functional characteristics of the peptide or polypeptide encoded by the nucleic acid) is used in the methods described herein, eg in the selection of an immunoglobulin single variable domain or other epitope binding domain, it is frequently advantageous to amplify or increase the copy number of the nucleic acids that encode the selected peptides or polypeptides. This provides an efficient way of obtaining sufficient quantities of nucleic acids and/or peptides or polypeptides for additional rounds of selection, using the methods described herein or other suitable methods, or for preparing additional repertoires (e.g., affinity maturation repertoires). Thus, in some embodiments, the methods of selecting epitope binding domains comprises using a display system (e.g., that links coding function of a nucleic acid and functional characteristics of the peptide or polypeptide encoded by the nucleic acid, such as phage display) and further comprises amplifying or increasing the copy number of a nucleic acid that encodes a selected peptide or polypeptide. Nucleic acids can be amplified using any suitable methods, such as by phage amplification, cell growth or polymerase chain reaction.

In one example, the methods employ a display system that links the coding function of a nucleic acid and physical, chemical and/or functional characteristics of the polypeptide encoded by the nucleic acid. Such a display system can comprise a plurality of replicable genetic packages, such as bacteriophage or cells (bacteria). The display system may comprise a library, such as a bacteriophage display library. Bacteriophage display is an example of a display system.

A number of suitable bacteriophage display systems (e.g., monovalent display and multivalent display systems) have been described. (See, e.g., Griffiths et al., U.S. Pat. No. 6,555,313 B1 (incorporated herein by reference); Johnson et al., U.S. Pat. No. 5,733,743 (incorporated herein by reference); McCafferty et al., U.S. Pat. No. 5,969,108 (incorporated herein by reference); Mulligan-Kehoe, U.S. Pat. No. 5,702,892 (Incorporated herein by reference); Winter, G. et al., Annu. Rev. Immunol. 12:433-455 (1994); Soumillion, P. et al., Appl. Biochem. Biotechnol. 47(2-3):175-189 (1994); Castagnoli, L. et al., Comb. Chem. High Throughput Screen, 4(2):121-133 (2001).) The peptides or polypeptides displayed in a bacteriophage display system can be displayed on any suitable bacteriophage, such as a filamentous phage (e.g., fd, M13, F1), a lytic phage (e.g., T4, T7, lambda), or an RNA phage (e.g., MS2), for example.

Generally, a library of phage that displays a repertoire of peptides or phagepolypeptides, as fusion proteins with a suitable phage coat protein (e.g., fd pIII protein), is produced or provided. The fusion protein can display the peptides or polypeptides at the tip of the phage coat protein, or if desired at an internal position. For example, the displayed peptide or polypeptide can be present at a position that is amino-terminal to domain 1 of pIII. (Domain 1 of pIII is also referred to as N1.) The displayed polypeptide can be directly fused to pIII (e.g., the N-terminus of domain 1 of pIII) or fused to pIII using a linker. If desired, the fusion can further comprise a tag (e.g., myc epitope, His tag). Libraries that comprise a repertoire of peptides or polypeptides that are displayed as fusion proteins with a phage coat protein, can be produced using any suitable methods, such as by introducing a library of phage vectors or phagemid vectors encoding the displayed peptides or polypeptides into suitable host bacteria, and culturing the resulting bacteria to produce phage (e.g., using a suitable helper phage or complementing plasmid if desired). The library of phage can be recovered from the culture using any suitable method, such as precipitation and centrifugation.

The display system can comprise a repertoire of peptides or polypeptides that contains any desired amount of diversity. For example, the repertoire can contain peptides or polypeptides that have amino acid sequences that correspond to naturally occurring polypeptides expressed by an organism, group of organisms, desired tissue or desired cell type, or can contain peptides or polypeptides that have random or randomized amino acid sequences. If desired, the polypeptides can share a common core or scaffold. For example, all polypeptides in the repertoire or library can be based on a scaffold selected from protein A, protein L, protein G, a fibronectin domain, an anticalin, CTLA4, a desired enzyme (e.g., a polymerase, a cellulase), or a polypeptide from the immunoglobulin superfamily, such as an antibody or antibody fragment (e.g., an antibody variable domain). The polypeptides in such a repertoire or library can comprise defined regions of random or randomized amino acid sequence and regions of common amino acid sequence. In certain embodiments, all or substantially all polypeptides in a repertoire are of a desired type, such as a desired enzyme (e.g., a polymerase) or a desired antigen-binding fragment of an antibody (e.g., human V_{H} or human V_{L}). In some embodiments, the polypeptide display system comprises a repertoire of polypeptides wherein each polypeptide comprises an antibody variable domain. For example, each polypeptide in the repertoire can contain a V_{H}, a V_{L} or an Fv (e.g., a single chain Fv).

Amino acid sequence diversity can be introduced into any desired region of a peptide or polypeptide or scaffold using any suitable method. For example, amino acid sequence diversity can be introduced into a target region, such as a complementarity determining region of an antibody variable domain or a hydrophobic domain, by preparing a library of nucleic acids that encode the diversified polypeptides using any suitable mutagenesis methods (e.g., low fidelity PCR, oligonucleotide-mediated or site directed mutagenesis, diversification using NNK codons) or any other suitable method. If desired, a region of a polypeptide to be diversified can be randomized. The size of the polypeptides that make up the repertoire is largely a matter of choice and uniform polypeptide size is not required. The polypeptides in the repertoire may have at least tertiary structure (form at least one domain).

Selection/Isolation/Recovery

An epitope binding domain or population of domains can be selected, isolated and/or recovered from a repertoire or library (e.g., in a display system) using any suitable method. For example, a domain is selected or isolated based on a selectable characteristic (e.g., physical characteristic, chemical characteristic, functional characteristic). Suitable selectable functional characteristics include biological activities of the peptides or polypeptides in the repertoire, for example, binding to a generic ligand (e.g., a superantigen), binding to a target ligand (e.g., an antigen, an epitope, a substrate), binding to an antibody (e.g., through an epitope expressed on a peptide or polypeptide), and catalytic activity. (See, e.g., Tomlinson et al., WO 99/20749; WO 01/57065; WO 99/58655.)

In some embodiments, the protease resistant peptide or polypeptide is selected and/or isolated from a library or repertoire of peptides or polypeptides in which substantially all domains share a common selectable feature. For example, the domain can be selected from a library or repertoire in which substantially all domains bind a common generic ligand, bind a common target ligand, bind (or are bound by) a common antibody, or possess a common catalytic activity. This type of selection is particularly useful for preparing a repertoire of domains that are based on a parental peptide or polypeptide that has a desired biological activity, for example, when performing affinity maturation of an immunoglobulin single variable domain.

Selection based on binding to a common generic ligand can yield a collection or population of domains that contain all or substantially all of the domains that were components of the original library or repertoire. For example, domains that bind a target ligand or a generic ligand, such as protein A, protein L or an antibody, can be selected, isolated and/or recovered by panning or using a suitable affinity matrix. Panning can be accomplished by adding a solution of ligand (e.g., generic ligand, target ligand) to a suitable vessel (e.g., tube, petri dish) and allowing the ligand to become deposited or coated onto the walls of the vessel. Excess ligand can be washed away and domains can be added to the vessel and the vessel maintained under conditions suitable for peptides or polypeptides to bind the immobilized ligand. Unbound domains can be washed away and bound domains can be recovered using any suitable method, such as scraping or lowering the pH, for example.

Suitable ligand affinity matrices generally contain a solid support or bead (e.g., agarose) to which a ligand is covalently or noncovalently attached. The affinity matrix can be combined with peptides or polypeptides (e.g., a repertoire that has been incubated with protease) using a batch process, a column process or any other suitable process under conditions suitable for binding of domains to the ligand on the matrix. domains that do not bind the affinity matrix can be washed away and bound domains can be eluted and recovered using any suitable method, such as elution with a lower pH buffer, with a mild denaturing agent (e.g., urea), or with a peptide or domain that competes for binding to the ligand. In one example, a biotinylated target ligand is combined with a repertoire under conditions suitable for domains in the repertoire to bind the target ligand. Bound domains are recovered using immobilized avidin or streptavidin (e.g., on a bead).

In some embodiments, the generic or target ligand is an antibody or antigen binding fragment thereof. Antibodies or antigen binding fragments that bind structural features of peptides or polypeptides that are substantially conserved in the peptides or polypeptides of a library or repertoire are particularly useful as generic ligands. Antibodies and antigen binding fragments suitable for use as ligands for isolating, selecting and/or recovering protease resistant peptides or polypeptides can be monoclonal or polyclonal and can be prepared using any suitable method.

Libraries/Repertoires

Libraries that encode and/or contain protease epitope binding domains can be prepared or obtained using any suitable method. A library can be designed to encode domains based on a domain or scaffold of interest (e.g., a domain selected from a library) or can be selected from another library using the methods described herein. For example, a library enriched in domains can be prepared using a suitable polypeptide display system.

Libraries that encode a repertoire of a desired type of domain can readily be produced using any suitable method. For example, a nucleic acid sequence that encodes a desired type of polypeptide (e.g., an immunoglobulin variable domain) can be obtained and a collection of nucleic acids that each contain one or more mutations can be prepared, for example by amplifying the nucleic acid using an error-prone polymerase chain reaction (PCR) system, by chemical mutagenesis (Deng et al., J. Biol. Chem., 269:9533 (1994)) or using bacterial mutator strains (Low et al., J. Mol. Biol., 260:359 (1996)).

In other embodiments, particular regions of the nucleic acid can be targeted for diversification. Methods for mutating selected positions are also well known in the art and include, for example, the use of mismatched oligonucleotides or degenerate oligonucleotides, with or without the use of PCR. For example, synthetic antibody libraries have been created by targeting mutations to the antigen binding loops. Random or semi-random antibody H3 and L3 regions have been appended to germline immunoblulin V gene segments to produce large libraries with unmutated framework regions (Hoogenboom and Winter (1992) supra; Nissim et al. (1994) supra; Griffiths et al. (1994) supra; DeKruif et al. (1995) supra). Such diversification has been extended to include some or all of the other antigen binding loops (Crameri et al. (1996) Nature Med., 2:100; Riechmann et al. (1995) Bio/Technology, 13:475; Morphosys, WO 97/08320, supra). In other embodiments, particular regions of the nucleic acid can be targeted for diversification by, for example, a two-step PCR strategy employing the product of the first PCR as a “mega-primer.” (See, e.g., Landt, O. et al., Gene 96:125-128 (1990).) Targeted diversification can also be accomplished, for example, by SOE PCR. (See, e.g., Horton, R. M. et al., Gene 77:61-68 (1989).)

Sequence diversity at selected positions can be achieved by altering the coding sequence which specifies the sequence of the polypeptide such that a number of possible amino acids (e.g., all 20 or a subset thereof) can be incorporated at that position. Using the IUPAC nomenclature, the most versatile codon is NNK, which encodes all amino acids as well as the TAG stop codon. The NNK codon may be used in order to introduce the required diversity. Other codons which achieve the same ends are also of use, including the NNN codon, which leads to the production of the additional stop codons TGA and TAA. Such a targeted approach can allow the full sequence space in a target area to be explored.

Some libraries comprise domains that are members of the immunoglobulin superfamily (e.g., antibodies or portions thereof). For example the libraries can comprise domains that have a known main-chain conformation. (See, e.g., Tomlinson et al., WO 99/20749.) Libraries can be prepared in a suitable plasmid or vector. As used herein, vector refers to a discrete element that is used to introduce heterologous DNA into cells for the expression and/or replication thereof. Any suitable vector can be used, including plasmids (e.g., bacterial plasmids), viral or bacteriophage vectors, artificial chromosomes and episomal vectors. Such vectors may be used for simple cloning and mutagenesis, or an expression vector can be used to drive expression of the library. Vectors and plasmids usually contain one or more cloning sites (e.g., a polylinker), an origin of replication and at least one selectable marker gene. Expression vectors can further contain elements to drive transcription and translation of a polypeptide, such as an enhancer element, promoter, transcription termination signal, signal sequences, and the like. These elements can be arranged in such a way as to be operably linked to a cloned insert encoding a polypeptide, such that the polypeptide is expressed and produced when such an expression vector is maintained under conditions suitable for expression (e.g., in a suitable host cell).

Cloning and expression vectors generally contain nucleic acid sequences that enable the vector to replicate in one or more selected host cells. Typically in cloning vectors, this sequence is one that enables the vector to replicate independently of the host chromosomal DNA and includes origins of replication or autonomously replicating sequences. Such sequences are well known for a variety of bacteria, yeast and viruses. The origin of replication from the plasmid pBR322 is suitable for most Gram-negative bacteria, the 2 micron plasmid origin is suitable for yeast, and various viral origins (e.g. SV40, adenovirus) are useful for cloning vectors in mammalian cells. Generally, the origin of replication is not needed for mammalian expression vectors, unless these are used in mammalian cells able to replicate high levels of DNA, such as COS cells.

Cloning or expression vectors can contain a selection gene also referred to as selectable marker. Such marker genes encode a protein necessary for the survival or growth of transformed host cells grown in a selective culture medium. Host cells not transformed with the vector containing the selection gene will therefore not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available in the growth media.

Suitable expression vectors can contain a number of components, for example, an origin of replication, a selectable marker gene, one or more expression control elements, such as a transcription control element (e.g., promoter, enhancer, terminator) and/or one or more translation signals, a signal sequence or leader sequence, and the like. Expression control elements and a signal or leader sequence, if present, can be provided by the vector or other source. For example, the transcriptional and/or translational control sequences of a cloned nucleic acid encoding an antibody chain can be used to direct expression.

A promoter can be provided for expression in a desired host cell. Promoters can be constitutive or inducible. For example, a promoter can be operably linked to a nucleic acid encoding an antibody, antibody chain or portion thereof, such that it directs transcription of the nucleic acid. A variety of suitable promoters for procaryotic (e.g., the β-lactamase and lactose promoter systems, alkaline phosphatase, the tryptophan (trp) promoter system, lac, tac, T3, T7 promoters for E. coli) and eucaryotic (e.g., simian virus 40 early or late promoter, Rous sarcoma virus long terminal repeat promoter, cytomegalovirus promoter, adenovirus late promoter, EG-1a promoter) hosts are available.

In addition, expression vectors typically comprise a selectable marker for selection of host cells carrying the vector, and, in the case of a replicable expression vector, an origin of replication. Genes encoding products which confer antibiotic or drug resistance are common selectable markers and may be used in procaryotic (e.g., β-lactamase gene (ampicillin resistance), Tet gene for tetracycline resistance) and eucaryotic cells (e.g., neomycin (G418 or geneticin), gpt (mycophenolic acid), ampicillin, or hygromycin resistance genes). Dihydrofolate reductase marker genes permit selection with methotrexate in a variety of hosts. Genes encoding the gene product of auxotrophic markers of the host (e.g., LEU2, URA3, HIS3) are often used as selectable markers in yeast. Use of viral (e.g., baculovirus) or phage vectors, and vectors which are capable of integrating into the genome of the host cell, such as retroviral vectors, are also contemplated.

Suitable expression vectors for expression in prokaryotic (e.g., bacterial cells such as E. coli) or mammalian cells include, for example, a pET vector (e.g., pET-12a, pET-36, pET-37, pET-39, pET-40, Novagen and others), a phage vector (e.g., pCANTAB 5 E, Pharmacia), pRIT2T (Protein A fusion vector, Pharmacia), pCDM8, pcDNA1.1/amp, pcDNA3.1, pRc/RSV, pEF-1 (Invitrogen, Carlsbad, Calif.), pCMV-SCRIPT, pFB, pSG5, pXT1 (Stratagene, La Jolla, Calif.), pCDEF3 (Goldman, L. A., et al., Biotechniques, 21:1013-1015 (1996)), pSVSPORT (GibcoBRL, Rockville, Md.), pEF-Bos (Mizushima, S., et al., Nucleic Acids Res., 18:5322 (1990)) and the like. Expression vectors which are suitable for use in various expression hosts, such as prokaryotic cells (E. coli), insect cells (Drosophila Schnieder S2 cells, Sf9), yeast (P. methanolica, P. pastoris, S. cerevisiae) and mammalian cells (eg, COS cells) are available.

Some examples of vectors are expression vectors that enable the expression of a nucleotide sequence corresponding to a polypeptide library member. Thus, selection with generic and/or target ligands can be performed by separate propagation and expression of a single clone expressing the polypeptide library member. As described above, a particular selection display system is bacteriophage display. Thus, phage or phagemid vectors may be used, for example vectors may be phagemid vectors which have an E. coli. origin of replication (for double stranded replication) and also a phage origin of replication (for production of single-stranded DNA). The manipulation and expression of such vectors is well known in the art (Hoogenboom and Winter (1992) supra; Nissim et al. (1994) supra). Briefly, the vector can contain a β-lactamase gene to confer selectivity on the phagemid and a lac promoter upstream of an expression cassette that can contain a suitable leader sequence, a multiple cloning site, one or more peptide tags, one or more TAG stop codons and the phage protein pIII. Thus, using various suppressor and non-suppressor strains of E. coli and with the addition of glucose, iso-propyl thio-β-D-galactoside (IPTG) or a helper phage, such as VCS M13, the vector is able to replicate as a plasmid with no expression, produce large quantities of the polypeptide library member only or product phage, some of which contain at least one copy of the polypeptide-pIII fusion on their surface.

Antibody variable domains may comprise a target ligand binding site and/or a generic ligand binding site. In certain embodiments, the generic ligand binding site is a binding site for a superantigen, such as protein A, protein L or protein G. The variable domains can be based on any desired variable domain, for example a human VH (e.g., V_{H} 1a, V_{H} 1b, V_{H} 2, V_{H} 3, V_{H} 4, V_{H} 5, V_{H} 6), a human Vλ (e.g., VλI, VλII, VλIII, VλIV, VλV, VλVI or Vκ1) or a human Vκ (e.g., Vκ2, Vκ3, Vκ4, Vκ5, Vκ6, Vκ7, Vκ8, Vκ9 or Vκ10).

A still further category of techniques involves the selection of repertoires in artificial compartments, which allow the linkage of a gene with its gene product. For example, a selection system in which nucleic acids encoding desirable gene products may be selected in microcapsules formed by water-in-oil emulsions is described in WO99/02671, WO00/40712 and Tawfik & Griffiths (1998) Nature Biotechnol 16(7), 652-6. Genetic elements encoding a gene product having a desired activity are compartmentalised into microcapsules and then transcribed and/or translated to produce their respective gene products (RNA or protein) within the microcapsules. Genetic elements which produce gene product having desired activity are subsequently sorted. This approach selects gene products of interest by detecting the desired activity by a variety of means.

Characterisation of the Epitope Binding Domains.

The binding of a domain to its specific antigen or epitope can be tested by methods which will be familiar to those skilled in the art and include ELISA. In one example, binding is tested using monoclonal phage ELISA.

Phage ELISA may be performed according to any suitable procedure: an exemplary protocol is set forth below.

Populations of phage produced at each round of selection can be screened for binding by ELISA to the selected antigen or epitope, to identify “polyclonal” phage antibodies. Phage from single infected bacterial colonies from these populations can then be screened by ELISA to identify “monoclonal” phage antibodies. It is also desirable to screen soluble antibody fragments for binding to antigen or epitope, and this can also be undertaken by ELISA using reagents, for example, against a C- or N-terminal tag (see for example Winter et al. (1994) Ann. Rev. Immunology 12, 433-55 and references cited therein.

The diversity of the selected phage monoclonal antibodies may also be assessed by gel electrophoresis of PCR products (Marks et al. 1991, supra; Nissim et al. 1994 supra), probing (Tomlinson et al., 1992) J. Mol. Biol. 227, 776) or by sequencing of the vector DNA.

Structure of dAbs

In the case that the dAbs are selected from V-gene repertoires selected for instance using phage display technology as herein described, then these variable domains comprise a universal framework region, such that is they may be recognised by a specific generic ligand as herein defined. The use of universal frameworks, generic ligands and the like is described in WO99/20749.

Where V-gene repertoires are used variation in polypeptide sequence may be located within the structural loops of the variable domains. The polypeptide sequences of either variable domain may be altered by DNA shuffling or by mutation in order to enhance the interaction of each variable domain with its complementary pair. DNA shuffling is known in the art and taught, for example, by Stemmer, 1994, Nature 370: 389-391 and U.S. Pat. No. 6,297,053, both of which are incorporated herein by reference. Other methods of mutagenesis are well known to those of skill in the art.

Scaffolds for Use in Constructing dAbs

i. Selection of the Main-Chain Conformation

The members of the immunoglobulin superfamily all share a similar fold for their polypeptide chain. For example, although antibodies are highly diverse in terms of their primary sequence, comparison of sequences and crystallographic structures has revealed that, contrary to expectation, five of the six antigen binding loops of antibodies (H1, H2, L1, L2, L3) adopt a limited number of main-chain conformations, or canonical structures (Chothia and Lesk (1987) J. Mol. Biol., 196: 901; Chothia et al. (1989) Nature, 342: 877). Analysis of loop lengths and key residues has therefore enabled prediction of the main-chain conformations of H1, H2, L1, L2 and L3 found in the majority of human antibodies (Chothia et al. (1992) J. Mol. Biol., 227: 799; Tomlinson et al. (1995) EMBO J., 14: 4628; Williams et al. (1996) J. Mol. Biol., 264: 220). Although the H3 region is much more diverse in terms of sequence, length and structure (due to the use of D segments), it also forms a limited number of main-chain conformations for short loop lengths which depend on the length and the presence of particular residues, or types of residue, at key positions in the loop and the antibody framework (Martin et al. (1996) J. Mol. Biol., 263: 800; Shirai et al. (1996) FEBS Letters, 399: 1).

The dAbs are advantageously assembled from libraries of domains, such as libraries of V_{H} domains and/or libraries of V_{L} domains. In one aspect, libraries of domains are designed in which certain loop lengths and key residues have been chosen to ensure that the main-chain conformation of the members is known. Advantageously, these are real conformations of immunoglobulin superfamily molecules found in nature, to minimise the chances that they are non-functional, as discussed above. Germline V gene segments serve as one suitable basic framework for constructing antibody or T-cell receptor libraries; other sequences are also of use. Variations may occur at a low frequency, such that a small number of functional members may possess an altered main-chain conformation, which does not affect its function.

Canonical structure theory is also of use to assess the number of different main-chain conformations encoded by ligands, to predict the main-chain conformation based on ligand sequences and to chose residues for diversification which do not affect the canonical structure. It is known that, in the human V_{κ} domain, the L1 loop can adopt one of four canonical structures, the L2 loop has a single canonical structure and that 90% of human V_{κ} domains adopt one of four or five canonical structures for the L3 loop (Tomlinson et al. (1995) supra); thus, in the V_{κ} domain alone, different canonical structures can combine to create a range of different main-chain conformations. Given that the Vλ domain encodes a different range of canonical structures for the L1, L2 and L3 loops and that V_{κ} and Vλ domains can pair with any V_{H} domain which can encode several canonical structures for the H1 and H2 loops, the number of canonical structure combinations observed for these five loops is very large. This implies that the generation of diversity in the main-chain conformation may be essential for the production of a wide range of binding specificities. However, by constructing an antibody library based on a single known main-chain conformation it has been found, contrary to expectation, that diversity in the main-chain conformation is not required to generate sufficient diversity to target substantially all antigens. Even more surprisingly, the single main-chain conformation need not be a consensus structure—a single naturally occurring conformation can be used as the basis for an entire library. Thus, in a one particular aspect, the dAbs possess a single known main-chain conformation.

The single main-chain conformation that is chosen may be commonplace among molecules of the immunoglobulin superfamily type in question. A conformation is commonplace when a significant number of naturally occurring molecules are observed to adopt it. Accordingly, in one aspect, the natural occurrence of the different main-chain conformations for each binding loop of an immunoglobulin domain are considered separately and then a naturally occurring variable domain is chosen which possesses the desired combination of main-chain conformations for the different loops. If none is available, the nearest equivalent may be chosen. The desired combination of main-chain conformations for the different loops may be created by selecting germline gene segments which encode the desired main-chain conformations. In one example, the selected germline gene segments are frequently expressed in nature, and in particular they may be the most frequently expressed of all natural germline gene segments.

In designing libraries the incidence of the different main-chain conformations for each of the six antigen binding loops may be considered separately. For H1, H2, L1, L2 and L3, a given conformation that is adopted by between 20% and 100% of the antigen binding loops of naturally occurring molecules is chosen. Typically, its observed incidence is above 35% (i.e. between 35% and 100%) and, ideally, above 50% or even above 65%. Since the vast majority of H3 loops do not have canonical structures, it is preferable to select a main-chain conformation which is commonplace among those loops which do display canonical structures. For each of the loops, the conformation which is observed most often in the natural repertoire is therefore selected. In human antibodies, the most popular canonical structures (CS) for each loop are as follows: H1—CS 1 (79% of the expressed repertoire), H2—CS 3 (46%), L1—CS 2 of V_{κ}(39%), L2—CS1 (100%), L3—CS1 of V_{κ}(36%) (calculation assumes a κ:λ ratio of 70:30, Hood et al. (1967) Cold Spring Harbor Symp. Quant. Biol., 48: 133). For H3 loops that have canonical structures, a CDR3 length (Kabat et al. (1991) Sequences of proteins of immunological interest, U.S. Department of Health and Human Services) of seven residues with a salt-bridge from residue 94 to residue 101 appears to be the most common. There are at least 16 human antibody sequences in the EMBL data library with the required H3 length and key residues to form this conformation and at least two crystallographic structures in the protein data bank which can be used as a basis for antibody modelling (2cgr and 1tet). The most frequently expressed germline gene segments that this combination of canonical structures are the V_{H} segment 3-23 (DP-47), the J_{H} segment JH4b, the V_{κ} segment O2/O12 (DPK9) and the J_{κ} segment J_{κ}1. V_{H} segments DP45 and DP38 are also suitable. These segments can therefore be used in combination as a basis to construct a library with the desired single main-chain conformation.

Alternatively, instead of choosing the single main-chain conformation based on the natural occurrence of the different main-chain conformations for each of the binding loops in isolation, the natural occurrence of combinations of main-chain conformations is used as the basis for choosing the single main-chain conformation. In the case of antibodies, for example, the natural occurrence of canonical structure combinations for any two, three, four, five, or for all six of the antigen binding loops can be determined. Here, the chosen conformation may be commonplace in naturally occurring antibodies and may be observed most frequently in the natural repertoire. Thus, in human antibodies, for example, when natural combinations of the five antigen binding loops, H1, H2, L1, L2 and L3, are considered, the most frequent combination of canonical structures is determined and then combined with the most popular conformation for the H3 loop, as a basis for choosing the single main-chain conformation.

Diversification of the Canonical Sequence

Having selected several known main-chain conformations or a single known main-chain conformation, dAbs can be constructed by varying the binding site of the molecule in order to generate a repertoire with structural and/or functional diversity. This means that variants are generated such that they possess sufficient diversity in their structure and/or in their function so that they are capable of providing a range of activities.

The desired diversity is typically generated by varying the selected molecule at one or more positions. The positions to be changed can be chosen at random or they may be selected. The variation can then be achieved either by randomisation, during which the resident amino acid is replaced by any amino acid or analogue thereof, natural or synthetic, producing a very large number of variants or by replacing the resident amino acid with one or more of a defined subset of amino acids, producing a more limited number of variants.

Various methods have been reported for introducing such diversity. Error-prone PCR (Hawkins et al. (1992) J. Mol. Biol., 226: 889), chemical mutagenesis (Deng et al. (1994) J. Biol. Chem., 269: 9533) or bacterial mutator strains (Low et al. (1996) J. Mol. Biol., 260: 359) can be used to introduce random mutations into the genes that encode the molecule. Methods for mutating selected positions are also well known in the art and include the use of mismatched oligonucleotides or degenerate oligonucleotides, with or without the use of PCR. For example, several synthetic antibody libraries have been created by targeting mutations to the antigen binding loops. The H3 region of a human tetanus toxoid-binding Fab has been randomised to create a range of new binding specificities (Barbas et al. (1992) Proc. Natl. Acad. Sci. USA, 89: 4457). Random or semi-random H3 and L3 regions have been appended to germline V gene segments to produce large libraries with unmutated framework regions (Hoogenboom & Winter (1992) J. Mol. Biol., 227: 381; Barbas et al. (1992) Proc. Natl. Acad. Sci. USA, 89: 4457; Nissim et al. (1994) EMBO J., 13: 692; Griffiths et al. (1994) EMBO J., 13: 3245; De Kruif et al. (1995) J. Mol. Biol., 248: 97). Such diversification has been extended to include some or all of the other antigen binding loops (Crameri et al. (1996) Nature Med., 2: 100; Riechmann et al. (1995) Bio/Technology, 13: 475; Morphosys, WO97/08320, supra).

Since loop randomisation has the potential to create approximately more than 10¹⁵ structures for H3 alone and a similarly large number of variants for the other five loops, it is not feasible using current transformation technology or even by using cell free systems to produce a library representing all possible combinations. For example, in one of the largest libraries constructed to date, 6×10¹⁰ different antibodies, which is only a fraction of the potential diversity for a library of this design, were generated (Griffiths et al. (1994) supra).

In a one embodiment, only those residues which are directly involved in creating or modifying the desired function of the molecule are diversified. For many molecules, the function will be to bind a target and therefore diversity should be concentrated in the target binding site, while avoiding changing residues which are crucial to the overall packing of the molecule or to maintaining the chosen main-chain conformation.

In one aspect, libraries of dAbs are used in which only those residues in the antigen binding site are varied. These residues are extremely diverse in the human antibody repertoire and are known to make contacts in high-resolution antibody/antigen complexes. For example, in L2 it is known that positions 50 and 53 are diverse in naturally occurring antibodies and are observed to make contact with the antigen. In contrast, the conventional approach would have been to diversify all the residues in the corresponding Complementarity Determining Region (CDR1) as defined by Kabat et al. (1991, supra), some seven residues compared to the two diversified in the library. This represents a significant improvement in terms of the functional diversity required to create a range of antigen binding specificities.

In nature, antibody diversity is the result of two processes: somatic recombination of germline V, D and J gene segments to create a naive primary repertoire (so called germline and junctional diversity) and somatic hypermutation of the resulting rearranged V genes. Analysis of human antibody sequences has shown that diversity in the primary repertoire is focused at the centre of the antigen binding site whereas somatic hypermutation spreads diversity to regions at the periphery of the antigen binding site that are highly conserved in the primary repertoire (see Tomlinson et al. (1996) J. Mol. Biol., 256: 813). This complementarity has probably evolved as an efficient strategy for searching sequence space and, although apparently unique to antibodies, it can easily be applied to other polypeptide repertoires. The residues which are varied are a subset of those that form the binding site for the target. Different (including overlapping) subsets of residues in the target binding site are diversified at different stages during selection, if desired.

In the case of an antibody repertoire, an initial ‘naive’ repertoire is created where some, but not all, of the residues in the antigen binding site are diversified. As used herein in this context, the term “naive” or “dummy” refers to antibody molecules that have no pre-determined target. These molecules resemble those which are encoded by the immunoglobulin genes of an individual who has not undergone immune diversification, as is the case with fetal and newborn individuals, whose immune systems have not yet been challenged by a wide variety of antigenic stimuli. This repertoire is then selected against a range of antigens or epitopes. If required, further diversity can then be introduced outside the region diversified in the initial repertoire. This matured repertoire can be selected for modified function, specificity or affinity.

It will be understood that the sequences described herein include sequences which are substantially identical, for example sequences which are at least 90% identical, for example which are at least 91%, or at least 92%, or at least 93%, or at least 94% or at least 95%, or at least 96%, or at least 97% or at least 98%, or at least 99% identical to the sequences described herein.

For nucleic acids, the term “substantial identity” indicates that two nucleic acids, or designated sequences thereof, when optimally aligned and compared, are identical, with appropriate nucleotide insertions or deletions, in at least about 80% of the nucleotides, usually at least about 90% to 95%, or at least about 98% to 99.5% of the nucleotides. Alternatively, substantial identity exists when the segments will hybridize under selective hybridization conditions, to the complement of the strand.

For nucleotide and amino acid sequences, the term “identical” indicates the degree of identity between two nucleic acid or amino acid sequences when optimally aligned and compared with appropriate insertions or deletions. Alternatively, substantial identity exists when the DNA segments will hybridize under selective hybridization conditions, to the complement of the strand.

The percent identity between two sequences is a function of the number of identical positions shared by the sequences (i.e., % identity=# of identical positions/total # of positions times 100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package, using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6.

By way of example, a polynucleotide sequence of the present invention may be identical to the reference sequence of SEQ ID NO: 1, that is be 100% identical, or it may include up to a certain integer number of nucleotide alterations as compared to the reference sequence. Such alterations are selected from the group consisting of at least one nucleotide deletion, substitution, including transition and transversion, or insertion, and wherein said alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in SEQ ID NO: 1 by the numerical percent of the respective percent identity (divided by 100) and subtracting that product from said total number of nucleotides in SEQ ID NO: 1, or:

nn≦xn−(xn·y),

wherein nn is the number of nucleotide alterations, xn is the total number of nucleotides in SEQ ID NO: 1, and y is 0.50 for 50%, 0.60 for 60%, 0.70 for 70%, 0.80 for 80%, 0.85 for 85%, 0.90 for 90%, 0.95 for 95%, 0.97 for 97% or 1.00 for 100%, and wherein any non-integer product of xn and y is rounded down to the nearest integer prior to subtracting it from xn. Alterations of the polynucleotide sequence of SEQ ID NO: 1 may create nonsense, missense or frameshift mutations in this coding sequence and thereby alter the polypeptide encoded by the polynucleotide following such alterations.

Similarly, in another example, a polypeptide sequence of the present invention may be identical to the reference sequence encoded by SEQ ID NO: 2, that is be 100% identical, or it may include up to a certain integer number of amino acid alterations as compared to the reference sequence such that the % identity is less than 100%. Such alterations are selected from the group consisting of at least one amino acid deletion, substitution, including conservative and non-conservative substitution, or insertion, and wherein said alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the polypeptide sequence encoded by SEQ ID NO: 2 by the numerical percent of the respective percent identity (divided by 100) and then subtracting that product from said total number of amino acids in the polypeptide sequence encoded by SEQ ID NO: 2, or:

na≦xa−(xa·y),

wherein na is the number of amino acid alterations, xa is the total number of amino acids in the polypeptide sequence encoded by SEQ ID NO: 2, and y is, for instance 0.70 for 70%, 0.80 for 80%, 0.85 for 85% etc., and wherein any non-integer product of xa and y is rounded down to the nearest integer prior to subtracting it from xa.

### EXAMPLES

### Example 1

### Design and Construction of a Triple-Targeting Antigen Binding Protein

An IgG-based molecule was constructed that was capable of binding to three different targets. The molecule comprised an antibody scaffold having independently selected Domain Antibodies (dAbs) against two different targets in place of the antibody variable domains, and a third dAb located on the C-terminus of the IgG heavy chain.

The triple targeting antigen binding protein exemplified herein is designated DMS4030. DMS4030 binds to VEGF via the VH dAb (DOM15-26-593) within the conventional IgG structure, whilst the VL dAb (DOM4-130-202) within the IgG binds to IL1-R1. The dAb appended to the C-terminus of the IgG binds to EGFR (DOM16-39-542).

### Example 2

### Cloning and Expression of DMS4030

To produce the DMS4030 molecule, the heavy chain expression plasmid used to make DMS4010 (pDMS4010-HC) was digested with BamHI and NheI to remove the cetuximab VH domain. The DOM15-26-593 dAb was amplified by PCR with the primers DT169 and DT093 to append BamHI and NheI to the 5′ and 3′ ends of the dAb ORF respectively.

DT093 GAATTATGGCTAGCGCTCGAGACGGTGACCAGGGT
DT169 GAATTATGGGATCCACCGGCGAGGTGCAGCTGTTGGTGTCTG

The PCR product was digested with BamHI and NheI and the purified fragment ligated into the cut pDMS4010-HC to created pDMS4030-HC.

After confirmation of the sequence of the plasmid, the DMS4030 molecule was expressed by transient transfection of HEK296/6E cells with both the pDMS4030-HC plasmid, and a light chain expression plasmid encoding the DOM4-130-54 dAb in the context of the Ck domain as described elsewhere (DMS2094). Transfections were carried out under standard conditions, and after 5 days the recombinant protein was harvested from the clarified supernatant by protein-A chromatography. Purified material was formulated in 10 mM Na citrate pH6, 10% (w/v) PEG-300, 5% sucrose (w/v) and maintained at 4° C.

The DMS4030 was estimated to have expressed at approximately 46 mg/l, and the quality of the material as determined by SDS-PAGE was comparable with conventional mAbs expressed and purified in a similar manner. This can be seen in FIG. 1.

The activity of the molecule was determined by independent assay of all three binding activities.

### Example 3

### VEGF Potency

VEGF potency was assessed by a VEGF receptor binding assay (RBA). This assay measures the binding of VEGF₁₆₅ to VEGF R2 (VEGF receptor) and the ability of test molecules to block this interaction. ELISA plates were coated overnight with VEGF receptor (R&D Systems, Cat No: 357-KD-050) (0.5 μg/ml final concentration in 0.2M sodium carbonate bicarbonate pH9.4), washed and blocked with 2% BSA in PBS. VEGF (R&D Systems, Cat No: 293-VE-050) and the test molecules (diluted in 0.1% BSA in 0.05% Tween 20™ PBS) were pre-incubated for one hour prior to addition to the plate (3 ng/ml VEGF final concentration). Binding of VEGF to VEGF receptor was detected using biotinylated anti-VEGF antibody (0.5 μg/ml final concentration) (R&D Systems, Cat No: BAF293) and a peroxidase conjugated anti-biotin secondary antibody (1:5000 dilution) (Stratech, Cat No: 200-032-096) and visualised at OD450 using a colorimetric substrate (Sure Blue TMB peroxidase substrate, KPL) after stopping the reaction with an equal volume of 1M HCl.

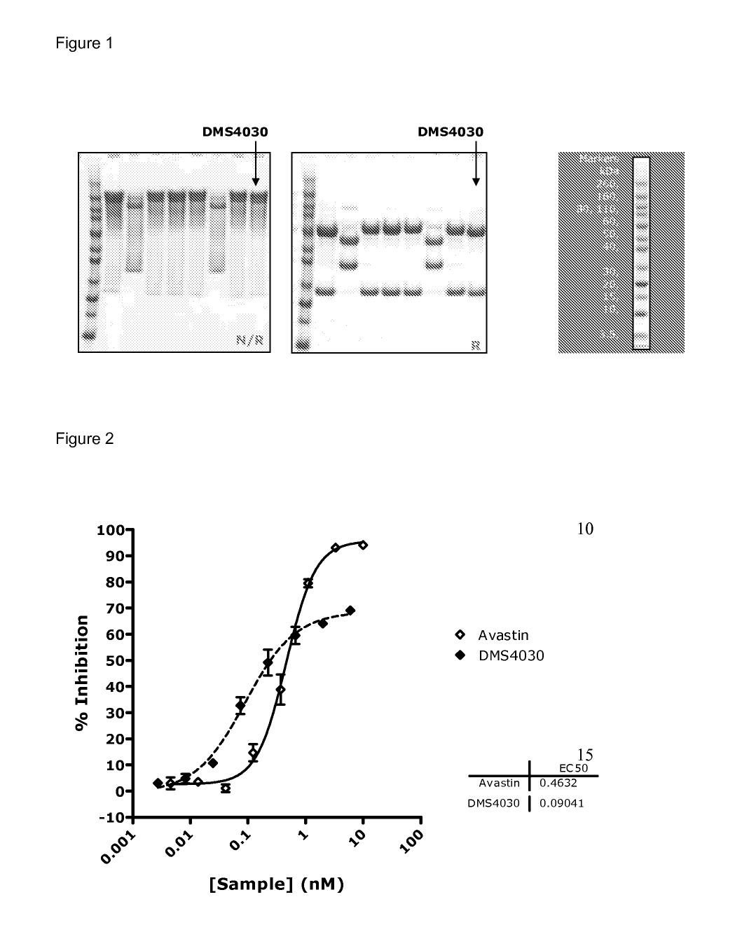

The results of this assay can be seen in FIG. 2, which shows that DMS4030 is able to block the binding of VEGF to VEGFR2 in this plate-based assay with an EC50 of 90 pM, although this figure is calculated on a curve that reaches only 70% maximal inhibition.

### Example 4

### IL1-R1 Bioassay

The ability of the DMS4030 to block signalling through IL1-1 was assessed in an IL-8 release assay in MRC-5 cells. The ability of test molecules to prevent human IL-1a binding to human IL1-R and neutralise IL-8 secretion was determined using human lung fibroblast MRC-5 cells. MRC-5 cells (ATCC, Cat.# CCL-171) were trypsinised then incubated with the test samples for one hour as a suspension. IL-1a (200 pg/ml final concentration) (R&D Systems cat no: 200-LA) was then added. After an overnight incubation IL-8 release was determined using an IL-8 quantification ELISA kit (R&D Systems) with anti-IL-8 coated ELISA plates, biotinylated anti-IL-8 and streptavidin-HRP. The assay readout is colourimetric absorbance at 450 nm and unknown IL-8 concentrations are interpolated using an IL-8 standard curve included in the assay.

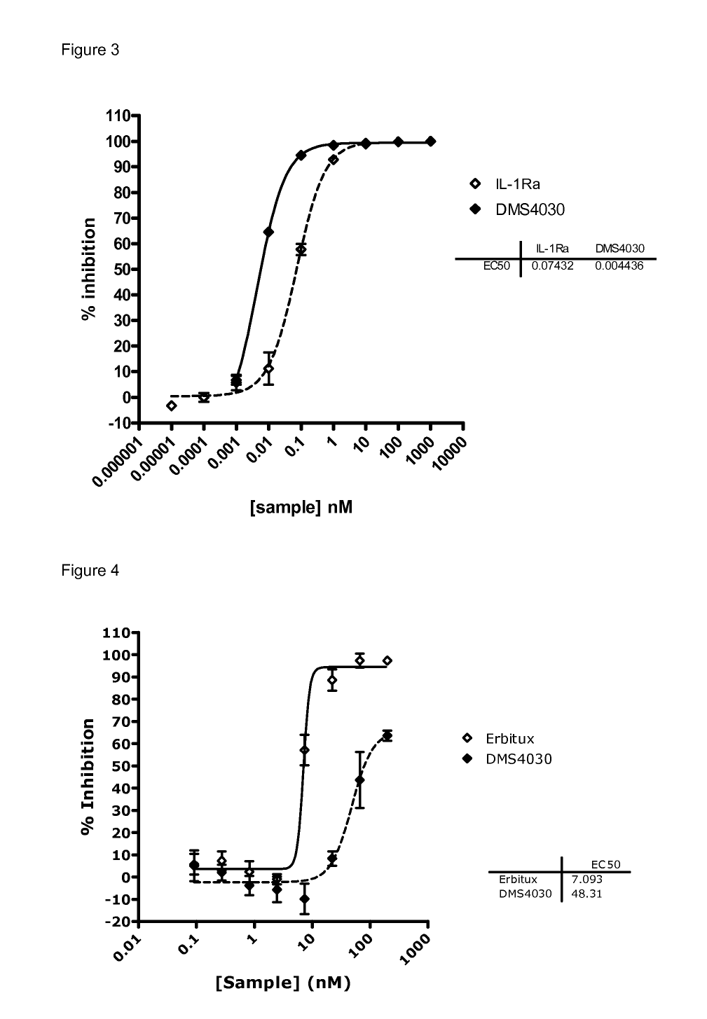

The results of this assay can be seen in FIG. 3, which shows DMS4030 was shown to be able to completely inhibit IL1 stimulated release of IL8 with an EC50 value of 4 pM in comparison with the natural IL1-R antagonist (IL1-Ra) which neutralised with an EC50 of 74 pM.

### Example 5

### EGFR Kinase Assay

DMS4030 was compared with a control anti-EGFR mAb (Erbitux) for its ability to inhibit EGF stimulated EGFR phosphorylation in EGFR positive A431 cells. Activation of EGFR expressed on the surface of A431 cells through its interaction with EGF leads to tyrosine kinase phosphorylation of the receptor. Reduction of EGFR tyrosine kinase phosphorylation was measured to determine potency of test molecules. A431 cells were allowed to adhere to 96 well tissue culture plates overnight then the test molecule was added and left for 1 hour and then incubated for 10 min with EGF (at 300 ng/ml) (R&D Systems catalogue number 236-EG). The cells were lysed and the lysed preparation transferred to ELISA plates coated with anti-EGFR antibody (at 1 ug/ml) (R&D Systems, cat #AF231). Both phosphorylated and non-phosphorylated EGFR present in the lysed cell solution was captured. After washing away unbound material phosphorylated EGFR was specifically detected using a HRP conjugated anti-phosphotyrosine antibody (1:2000 dilution) (Upstate Biotechnology, cat #16-105). Binding was visualised using a colorimetric substrate.

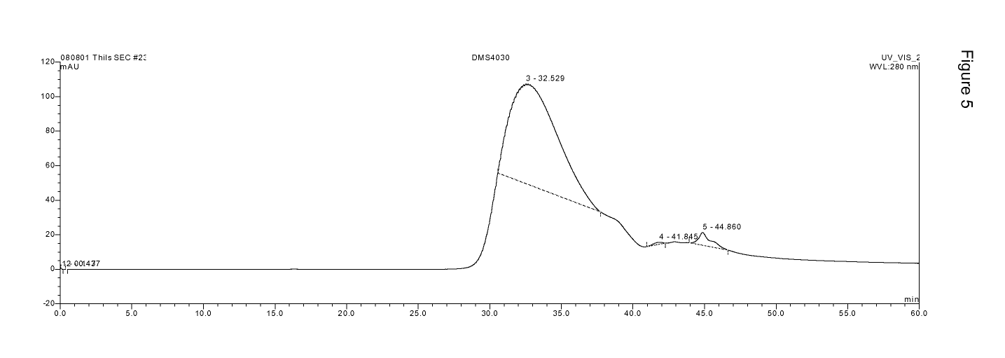

The results of this assay can be seen in FIG. 4, which shows DMS4030 inhibited EGFR phosphorylation to a maximum of ·60% with an EC50 of 48 pM, compared with 100% inhibition (EC50 7 pM) for the anti-EGFR mAb.

The biophysical properties of the DMS4030 molecule were assessed by determination of the SEC profile. The DMS4030 was applied onto a Superdex-200 10/30 HR column (attached to an Akta Express FPLC system) pre-equilibrated and running in PBS at 0.5 ml/min. The SEC profile for DMS4030 is shown in FIG. 5, with a broad single major peak and some lower molecular weight contaminants emerging after the main peak

### Example 6

### Stoichiometry Assessment of Antigen Binding Proteins (Using Biacore™)

This example is prophetic. It provides guidance for carrying out an additional assay in which the antigen binding proteins of the invention can be tested,

Anti-human IgG is immobilised onto a CM5 biosensor chip by primary amine coupling. Antigen binding proteins are captured onto this surface after which a single concentration of the antigen (e.g. TNFα or VEGF) is passed over, this concentration is enough to saturate the binding surface and the binding signal observed reached full R-max. Stoichiometries are then calculated using the given formula:

Stoich=Rmax*Mw (ligand)/Mw (analyte)*R (ligand immobilised or captured)

Where the stoichiometries are calculated for more than one analyte binding at the same time, the different antigens are passed over sequentially at the saturating antigen concentration and the stoichometries calculated as above. The work can be carried out on the Biacore 3000, at 25° C. using HBS-EP running buffer.

Sequences

| | Sequence identifier (SEQ ID NO) | |
|---|---|---|
| | amino acid | |
| Description | sequence | DNA sequence |
| DMS4030-Heavy chain (DOM15-26-593- | 2 | 1 |
| DOM16-39-542) | | |
| DMS4030-Light chain (DOM4-130-54) | 4 | 3 |
| mAb heavy chain constant region | 5 | |
| mAb light chain constant region | 6 | |
| DOM15-26-593 | 7 | |
| DOM16-39-542 | 8 | |
| DOM4-130-54 | 9 | |
| GS linker | 10 | |
| TVAAPSGS linker | 11 | |
| PAS linker | 12 | |
| G₄S linker | 13 | |
| PAVPPP linker | 14 | |
| TVSDVP linker | 15 | |
| TGLDSP linker | 16 | |
| TVAAPS linker | 17 | |
| GS(TVAAPSGS)₁ Linker | 18 | |
| GS(TVAAPSGS)₂ Linker | 19 | |
| GS(TVAAPSGS)₃ Linker | 20 | |
| GS(TVAAPSGS)₄ Linker | 21 | |
| GS(TVAAPSGS)₅ Linker | 22 | |
| GS(TVAAPSGS)₆ Linker | 23 | |
| (PAS)₁GS Linker | 24 | |
| (PAS)₂GS Linker | 25 | |
| (PAS)₃GS Linker | 26 | |
| (G₄S)₁ Linker | 27 | |
| (G₄S)₂ Linker | 28 | |
| (G₄S)₃ Linker | 29 | |
| (PAVPPP)₁GS Linker | 30 | |
| (PAVPPP)₂GS Linker | 31 | |
| (PAVPPP)₃GS Linker | 32 | |
| (TVSDVP)₁GS Linker | 33 | |
| (TVSDVP)₂GS Linker | 34 | |
| (TVSDVP)₃GS Linker | 35 | |
| (TGLDSP)₁GS Linker | 36 | |
| (TGLDSP)₂GS Linker | 37 | |
| (TGLDSP)₃GS Linker | 38 | |
| Anti-VEGFR2 adnectin | 39 | |
| Anti-TNFalpha adnectin | 40 | |
| Anti-VEGF Anticalin | 41 | |
| (TVAAPS)₂(GS)₁ Linker | 42 | |
| (TVAAPS)₃(GS)₁ Linker | 43 | |
| GSTVAAPS Linker | 44 | |

SEQ ID NO: 1 (pDMS4030-HC (pDOM15-26-593-DOM16-39-542))
ATGGAGACCGACACCCTGCTGCTGTGGGTGCTGCTGCTGTGGGTGCCCGGATCCACCGGCGAGGTGCAG
CTGTTGGTGTCTGGGGGAGGCTTGGTACAGCCTGGGGGGTCCCTGCGTCTCTCCTGTGCAGCCTCCGGA
TTCACCTTTAAGGCTTATCCGATGATGTGGGTCCGCCAGGCTCCAGGGAAGGGTCTAGAGTGGGTTTCA
GAGATTTCGCCTTCGGGTTCTTATACATACTACGCAGACTCCGTGAAGGGCCGGTTCACCATCTCCCGC
GACAATTCCAAGAACACGCTGTATCTGCAAATGAACAGCCTGCGTGCCGAGGACACCGCGGTATATTAC
TGTGCGAAAGATCCTCGGAAGTTAGACTACTGGGGTCAGGGAACCCTGGTCACCGTCTCGAGCGCTAGC
ACCAAGGGCCCCAGCGTGTTCCCCCTGGCCCCCAGCAGCAAGAGCACCAGCGGCGGCACAGCCGCCCTG
GGCTGCCTGGTGAAGGACTACTTCCCCGAGCCTGTGACCGTGTCCTGGAATAGCGGAGCCCTGACCTCC
GGCGTGCACACCTTCCCCGCCGTGCTGCAGAGCAGCGGCCTGTACTCCCTGAGCAGCGTGGTGACCGTG
CCCAGCAGCAGCCTGGGCACCCAGACCTACATCTGCAACGTGAACCACAAGCCCAGCAACACCAAAGTG
GACAAGAAAGTGGAGCCCAAGAGCTGCGATAAGACCCACACCTGCCCCCCCTGCCCTGCCCCCGAGCTG
CTGGGCGGACCTAGCGTGTTCCTGTTCCCCCCCAAGCCTAAGGACACCCTGATGATCAGCAGGACCCCC
GAAGTGACCTGCGTGGTGGTGGATGTGAGCCACGAGGACCCTGAAGTGAAGTTCAACTGGTACGTGGAC
GGCGTGGAAGTGCACAACGCCAAGACCAAGCCCAGAGAGGAGCAGTACAACAGCACCTACCGCGTGGTG
TCTGTGCTGACCGTGCTGCACCAGGATTGGCTGAACGGCAAGGAGTACAAGTGCAAAGTGAGCAACAAG
GCCCTGCCTGCCCCTATCGAGAAAACCATCAGCAAGGCCAAGGGCCAGCCTAGAGAGCCCCAGGTCTAC
ACCCTGCCTCCCTCCAGAGATGAGCTGACCAAGAACCAGGTGTCCCTGACCTGTCTGGTGAAGGGCTTC
TACCCCAGCGACATCGCCGTGGAGTGGGAGAGCAACGGCCAGCCCGAGAACAACTACAAGACCACCCCC
CCTGTGCTGGACAGCGATGGCAGCTTCTTCCTGTACTCCAAGCTGACCGTGGACAAGAGCAGATGGCAG
CAGGGCAACGTGTTCAGCTGCAGCGTGATGCACGAGGCCCTGCACAATCACTACACCCAGAAGAGTCTG
AGCCTGTCCCCTGGCAAGTCGACCGGTGACATCCAGATGACCCAGTCTCCATCAAGCCTGAGCGCCAGC
GTGGGCGACAGAGTGACCATCACCTGCCGGGCCAGCCAGTGGATCGGCAACCTGCTGGACTGGTATCAG
CAGAAGCCCGGCAAGGCCCCCAAGCTGCTGATCTACTACGCCAGCTTCCTGCAGAGCGGCGTGCCCAGC
CGGTTTAGCGGCAGCGGCTACGGCACCGACTTCACCCTGACCATCAGCAGCCTGCAGCCCGAGGACTTC
GCCACCTACTACTGCCAGCAGGCCAACCCTGCCCCCCTGACCTTCGGCCAGGGGACCAAGGTGGAAATC
AAACGGTAA
SEQ ID NO: 2 (pDMS4030-HC (pDOM15-26-593-DOM16-39-542))
EVQLLVSGGGLVQPGGSLRLSCAASGFTFKAYPMMWVRQAPGKGLEWVSEISPSGSYTYYADSVKGRFT
ISRDNSKNTLYLQMNSLRAEDTAVYYCAKDPRKLDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSN
TKVDKKVEPKSCDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSFFLYSKLTVDKS
RWQQGNVFSCSVMHEALHNHYTQKSLSLSPGKSTGDIQMTQSPSSLSASVGDRVTITCRASQWIGNLLD
WYQQKPGKAPKLLIYYASFLQSGVPSRFSGSGYGTDFTLTISSLQPEDFATYYCQQANPAPLTFGQGTK
VEIKR
SEQ ID NO: 3 (pDMS4030-LC (pDOM39-DOM4-130-54))
GACATCCAGATGACCCAGTCTCCATCCTCCCTGTCTGCATCTGTAGGAGACCGTGTCACCATCACTTGC
CGGGCAAGTCAGGATATTTACCTGAATTTAGACTGGTATCAGCAGAAACCAGGGAAAGCCCCTAAGCTC
CTGATCAATTTTGGTTCCGAGTTGCAAAGTGGTGTCCCATCACGTTTCAGTGGCAGTGGATATGGGACA
GATTTCACTCTCACCATCAGCAGTCTGCAACCTGAAGATTTCGCTACGTACTACTGTCAACCGTCTTTT
TACTTCCCTTATACGTTCGGCCAAGGGACCAAGGTGGAAATCAAACGGACGGTGGCCGCCCCCAGCGTG
TTCATCTTCCCCCCCAGCGATGAGCAGCTCAAGAGCGGCACCGCCAGCGTGGTGTGTCTGCTGAACAAC
TTCTACCCCCGGGAGGCCAAAGTGCAGTGGAAAGTGGACAACGCCCTGCAGAGCGGCAACAGCCAGGAG
AGCGTGACCGAGCAGGACAGCAAGGACTCCACCTACAGCCTGAGCAGCACCCTGACCCTGAGCAAGGCC
GACTACGAGAAGCACAAAGTGTACGCCTGCGAAGTGACCCACCAGGGCCTGTCCAGCCCCGTGACCAAG
AGCTTCAACCGGGGCGAGTGCTGA
SEQ ID NO: 4 (pDMS4030-LC (pDOM39-DOM4-130-54)
DIQMTQSPSSLSASVGDRVTITCRASQDIYLNLDWYQQKPGKAPKLLINFGSELQSGVPSRFSGSGYGT
DFTLTISSLQPEDFATYYCQPSFYFPYTFGQGTKVEIKRTVAAPSVFIFPPSDEQLKSGTASVVCLLNN
FYPREAKVQWKVDNALQSGNSQESVTEQDSKDSTYSLSSTLTLSKADYEKHKVYACEVTHQGLSSPVTK
SFNRGEC
SEQ ID NO: 5 (mAb heavy chain constant region)
ASTKGPSVFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPKDTLMISR
TPEVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLSLSPGK
SEQ ID NO: 6 (mAb light chain constant region)
TVAAPSVFIFPPSDEQLKSGTASVVCLLNNFYPREAKVQWKVDNALQSGNSQESVTEQDSKDSTYSLSS
TLTLSKADYEKHKVYACEVTHQGLSSPVTKSFNRGEC
SEQ ID NO: 7 (DOM15-26-593)
EVQLLVSGGGLVQPGGSLRLSCAASGFTFKAYPMMWVRQAPGKGLEWVSEISPSGSYTYYADSVKGRFT
ISRDNSKNTLYLQMNSLRAEDTAVYYCAKDPRKLDYWGQGTLVTVSS
SEQ ID NO: 8 (DOM16-39-542)
DIQMTQSPSSLSASVGDRVTITCRASQWIGNLLDWYQQKPGKAPKLLIYYASFLQSGVPSRFSGSGYGT
DFTLTISSLQPEDFATYYCQQANPAPLTFGQGTKVEIKR
SEQ ID NO: 9 (DOM4-130-54)
DIQMTQSPSSLSASVGDRVTITCRASQDIYLNLDWYQQKPGKAPKLLINFGSELQSGVPSRFSGSGYGT
DFTLTISSLQPEDFATYYCQPSFYFPYTFGQGTKVEIKR
SEQ ID NO: 10
GS
SEQ ID NO: 11
TVAAPSGS
SEQ ID NO: 12
PAS
SEQ ID NO: 13
GGGGS
SEQ ID NO: 14
PAVPPP
SEQ ID NO: 15
TVSDVP
SEQ ID NO: 16
TGLDSP
SEQ ID NO: 17
TVAAPS
SEQ ID NO: 18
GSTVAAPSGS
SEQ ID NO: 19
GSTVAAPSGSTVAAPSGS
SEQ ID NO: 20
GSTVAAPSGSTVAAPSGSTVAAPSGS
SEQ ID NO: 21
GSTVAAPSGSTVAAPSGSTVAAPSGSTVAAPSGS
SEQ ID NO: 22
GSTVAAPSGSTVAAPSGSTVAAPSGSTVAAPSGSTVAAPSGS
SEQ ID NO: 23
GSTVAAPSGSTVAAPSGSTVAAPSGSTVAAPSGSTVAAPSGSTVAAPSGS
SEQ ID NO: 24
PASGS
SEQ ID NO: 25
PASPASGS
SEQ ID NO: 26
PASPASPASGS
SEQ ID NO: 27
GGGGS
SEQ ID NO: 28
GGGGSGGGGS
SEQ ID NO: 29
GGGGSGGGGSGGGGS
SEQ ID NO: 30
PAVPPPGS
SEQ ID NO: 31
PAVPPPPAVPPPGS
SEQ ID NO: 32
PAVPPPPAVPPPPAVPPPGS
SEQ ID NO: 33
TVSDVPGS
SEQ ID NO: 34
TVSDVPTVSDVPGS
SEQ ID NO: 35
TVSDVPTVSDVPTVSDVPGS
SEQ ID NO: 36
TGLDSPGS
SEQ ID NO: 37
TGLDSPTGLDSPGS
SEQ ID NO: 38
TGLDSPTGLDSPTGLDSPGS
SEQ ID NO: 39 (anti-VEGFR2 adnectin)
EVVAATPTSLLISWRHPHFPTRYYRITYGETGGNSPVQEFTVPLQPPTATISGLKPGVDYTI
TVYAVTDGRNGRLLSIPISINYRT
SEQ ID NO: 40 (anti-TNFalpha adnectin)
VSDVPRDLEVVAATPTSLLISWDTHNAYNGYYRITYGETGGNSPVREFTVPHPEVTATISGL
KPGVDDTITVYAVTNHHMPLRIFGPISINHRT
SEQ ID NO: 41 (anti-VEGF Anticalin)
DGGGIRRSMSGTWYLKAMTVDREFPEMNLESVTPMTLTLLKGHNLEAKVTMLISGRCQEVKAVLGRTKE
RKKYTADGGKHVAYIIPSAVRDHVIFYSEGQLHGKPVRGVKLVGRDPKNNLEALEDFEKAAGARGLSTE
SILIPRQSETCSPG
SEQ ID NO: 42
TVAAPSTVAAPSGS
SEQ ID NO: 43
TVAAPSTVAAPSTVAAPSGS
SEQ ID NO: 44
GSTVAAPS

1-21. (canceled)

22. An antigen-binding protein comprising an immunoglobulin heavy chain and an immunoglobulin light chain, wherein the heavy chain comprises an epitope binding domain linked to the n-terminus of CH1-CH2-CH3, and the light chain comprises an epitope binding domain linked to the n-terminus of CL, wherein one or more epitope binding domains are linked to the c-terminus of one or both of the immunoglobulin heavy chain or immunoglobulin light chain.

23. The antigen binding protein according to claim 22 wherein an epitope binding domain is linked to the c-terminus of the heavy chain.

24. The antigen binding protein according to claim 22 wherein an epitope binding domain is linked to the c-terminus of the light chain.

25. The antigen-binding protein according to claim 22 wherein at least one epitope binding domain is an immunoglobulin single variable domain.

26. The antigen-binding protein according to claim 25 wherein the immunoglobulin single variable domain is a human dAb.

27. The antigen-binding protein of claim 22 wherein the binding protein has specificity for more than one antigen.

28. The antigen-binding protein of claim 22 wherein the binding protein has specificity for three antigens.

29. The antigen-binding protein according to claim 22 wherein at least one epitope binding domain is capable of binding VEGF or VEGFR2 or TNFα or IL1R1.

30. The antigen-binding protein according to claim 22 wherein at least one of the epitope binding domains is linked to the mAb scaffold by a linker comprising from 1 to 150 amino acids.

31. The antigen-binding protein according to claim 30 wherein at least one of the epitope binding domains is linked to the mAb scaffold with a linker selected from any one of those set out in SEQ ID NO:10-38, or any multiple or combination thereof.

32. The antigen-binding protein according to claim 22 wherein at least one of the epitope binding domains binds human serum albumin.

33. A polynucleotide sequence encoding a heavy chain or light chain of the antigen-binding protein according to any one of claim 22.

34. A recombinant transformed or transfected host cell comprising one or more polynucleotide sequences encoding an antigen-binding protein of claim 22.

35. A method for the production of an antigen-binding protein according to claim 22 which method comprises the step of culturing a recombinant transformed or transfected host cell comprising one or more polynucleotide sequences encoding an antigen-binding protein of claim 22 and isolating the antigen-binding protein.

36. A pharmaceutical composition comprising an antigen-binding protein of any one of claim 22 and a pharmaceutically acceptable carrier.

37. The antigen-binding protein according to any one of claim 22 for use in medicine.


SEQUENCE LISTING (44 sequences)

SEQ ID NO: 1 - DNA, 1734 bp, Artificial Sequence
  Fusion proteins
atggagaccgacaccctgctgctgtgggtgctgctgctgtgggtgcccggatccaccggcgaggtgcagctgttggtgtctgggggaggcttggtacagcctggggggtccctgcgtctctcctgtgcagcctccggattcacctttaaggcttatccgatgatgtgggtccgccaggctccagggaagggtctagagtgggtttcagagatttcgccttcgggttcttatacatactacgcagactccgtgaagggccggttcaccatctcccgcgacaattccaagaacacgctgtatctgcaaatgaacagcctgcgtgccgaggacaccgcggtatattactgtgcgaaagatcctcggaagttagactactggggtcagggaaccctggtcaccgtctcgagcgctagcaccaagggccccagcgtgttccccctggcccccagcagcaagagcaccagcggcggcacagccgccctgggctgcctggtgaaggactacttccccgagcctgtgaccgtgtcctggaatagcggagccctgacctccggcgtgcacaccttccccgccgtgctgcagagcagcggcctgtactccctgagcagcgtggtgaccgtgcccagcagcagcctgggcacccagacctacatctgcaacgtgaaccacaagcccagcaacaccaaagtggacaagaaagtggagcccaagagctgcgataagacccacacctgccccccctgccctgcccccgagctgctgggcggacctagcgtgttcctgttcccccccaagcctaaggacaccctgatgatcagcaggacccccgaagtgacctgcgtggtggtggatgtgagccacgaggaccctgaagtgaagttcaactggtacgtggacggcgtggaagtgcacaacgccaagaccaagcccagagaggagcagtacaacagcacctaccgcgtggtgtctgtgctgaccgtgctgcaccaggattggctgaacggcaaggagtacaagtgcaaagtgagcaacaaggccctgcctgcccctatcgagaaaaccatcagcaaggccaagggccagcctagagagccccaggtctacaccctgcctccctccagagatgagctgaccaagaaccaggtgtccctgacctgtctggtgaagggcttctaccccagcgacatcgccgtggagtgggagagcaacggccagcccgagaacaactacaagaccaccccccctgtgctggacagcgatggcagcttcttcctgtactccaagctgaccgtggacaagagcagatggcagcagggcaacgtgttcagctgcagcgtgatgcacgaggccctgcacaatcactacacccagaagagtctgagcctgtcccctggcaagtcgaccggtgacatccagatgacccagtctccatcaagcctgagcgccagcgtgggcgacagagtgaccatcacctgccgggccagccagtggatcggcaacctgctggactggtatcagcagaagcccggcaaggcccccaagctgctgatctactacgccagcttcctgcagagcggcgtgcccagccggtttagcggcagcggctacggcaccgacttcaccctgaccatcagcagcctgcagcccgaggacttcgccacctactactgccagcaggccaaccctgcccccctgaccttcggccaggggaccaaggtggaaatcaaacggtaa

SEQ ID NO: 2 - protein, 551 aa, Artificial Sequence
  Fusion proteins
EVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSSASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGKSTGDIQMTQSPSSLSASVGDRVTITCRASQWIGNLLDWYQQKPGKAPKLLIYYASFLQSGVPSRFSGSGYGTDFTLTISSLQPEDFATYYCQQANPAPLTFGQGTKVEIKR

SEQ ID NO: 3 - DNA, 645 bp, Artificial Sequence
  Fusion proteins
gacatccagatgacccagtctccatcctccctgtctgcatctgtaggagaccgtgtcaccatcacttgccgggcaagtcaggatatttacctgaatttagactggtatcagcagaaaccagggaaagcccctaagctcctgatcaattttggttccgagttgcaaagtggtgtcccatcacgtttcagtggcagtggatatgggacagatttcactctcaccatcagcagtctgcaacctgaagatttcgctacgtactactgtcaaccgtctttttacttcccttatacgttcggccaagggaccaaggtggaaatcaaacggacggtggccgcccccagcgtgttcatcttcccccccagcgatgagcagctcaagagcggcaccgccagcgtggtgtgtctgctgaacaacttctacccccgggaggccaaagtgcagtggaaagtggacaacgccctgcagagcggcaacagccaggagagcgtgaccgagcaggacagcaaggactccacctacagcctgagcagcaccctgaccctgagcaaggccgactacgagaagcacaaagtgtacgcctgcgaagtgacccaccagggcctgtccagccccgtgaccaagagcttcaaccggggcgagtgctga

SEQ ID NO: 4 - protein, 214 aa, Artificial Sequence
  Fusion proteins
DIQMTQSPSSLSASVGDRVTITCRASQDIYLNLDWYQQKPGKAPKLLINFGSELQSGVPSRFSGSGYGTDFTLTISSLQPEDFATYYCQPSFYFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 324 aa, Artificial Sequence
  Humanised
ASTKGPSVFPLAPSSKSTSGGTAALGCLVKDYFPEPVTVSWNSGALTSGVHTFPAVLQSSGSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSYVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFKKLTVDKSRWQQGNVFSCSVMHEALHNHYTQKSLSLSPGK

SEQ ID NO: 6 - protein, 106 aa, Artificial Sequence
  Humanised
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 116 aa, Homo Sapiens
EVQLLVSGGGLVQPGGSLRLSCAASGFTFKAYPMMWVRQAPGKGLEWVSEISPSGSYTYYADSVKGRFTISRDNSKNTLYLQMNSLRAEDTAVYYCAKDPRKLDYWGQGTLVTVSS

SEQ ID NO: 8 - protein, 108 aa, Homo Sapiens
DIQMTQSPSSLSASVGDRVTITCRASQWIGNLLDWYQQKPGKAPKLLIYYASFLQSGVPSRFSGSGYGTDFTLTISSLQPEDFATYYCQQANPAPLTFGQGTKVEIKR

SEQ ID NO: 9 - protein, 108 aa, Homo Sapiens
DIQMTQSPSSLSASVGDRVTITCRASQDIYLNLDWYQQKPGKAPKLLINFGSELQSGVPSRFSGSGYGTDFTLTISSLQPEDFATYYCQPSFYFPYTFGQGTKVEIKR

SEQ ID NO: 10 - protein, 2 aa, Artificial Sequence
  Linker
GS

SEQ ID NO: 11 - protein, 8 aa, Artificial Sequence
  Linker
TVAAPSGS

SEQ ID NO: 12 - protein, 3 aa, Artificial Sequence
  Linker
PAS

SEQ ID NO: 13 - protein, 5 aa, Artificial Sequence
  Linker
GGGGS

SEQ ID NO: 14 - protein, 6 aa, Artificial Sequence
  Linker
PAVPPP

SEQ ID NO: 15 - protein, 6 aa, Artificial Sequence
  Linker
TVSDVP

SEQ ID NO: 16 - protein, 6 aa, Artificial Sequence
  Linker
TGLDSP

SEQ ID NO: 17 - protein, 6 aa, Artificial Sequence
  Linker
TVAAPS

SEQ ID NO: 18 - protein, 10 aa, Artificial Sequence
  Linker
GSTVAAPSGS

SEQ ID NO: 19 - protein, 18 aa, Artificial Sequence
  Linker
GSTVAAPSGSTVAAPSGS

SEQ ID NO: 20 - protein, 26 aa, Artificial Sequence
  Linker
GSTVAAPSGSTVAAPSGSTVAAPSGS

SEQ ID NO: 21 - protein, 34 aa, Artificial Sequence
  Linker
GSTVAAPSGSTVAAPSGSTVAAPSGSTVAAPSGS

SEQ ID NO: 22 - protein, 42 aa, Artificial Sequence
  Linker
GSTVAAPSGSTVAAPSGSTVAAPSGSTVAAPSGSTVAAPSGS

SEQ ID NO: 23 - protein, 50 aa, Artificial Sequence
  Linker
GSTVAAPSGSTVAAPSGSTVAAPSGSTVAAPSGSTVAAPSGSTVAAPSGS

SEQ ID NO: 24 - protein, 5 aa, Artificial Sequence
  Linker
PASGS

SEQ ID NO: 25 - protein, 6 aa, Artificial Sequence
  Linker
PDASGS

SEQ ID NO: 26 - protein, 7 aa, Artificial Sequence
  Linker
PDDASGS

SEQ ID NO: 27 - protein, 5 aa, Artificial Sequence
  Linker
GGGGS

SEQ ID NO: 28 - protein, 10 aa, Artificial Sequence
  Linker
GGGGSGGGGS

SEQ ID NO: 29 - protein, 15 aa, Artificial Sequence
  Linker
GGGGSGGGGSGGGGS

SEQ ID NO: 30 - protein, 8 aa, Artificial Sequence
  Linker
PAVPPPGS

SEQ ID NO: 31 - protein, 14 aa, Artificial Sequence
  Linker
PAVPPPPAVPPPGS

SEQ ID NO: 32 - protein, 20 aa, Artificial Sequence
  Linker
PAVPPPPAVPPPPAVPPPGS

SEQ ID NO: 33 - protein, 8 aa, Artificial Sequence
  Linker
TVSDVPGS

SEQ ID NO: 34 - protein, 14 aa, Artificial Sequence
  Linker
TVSDVPTVSDVPGS

SEQ ID NO: 35 - protein, 20 aa, Artificial Sequence
  Linker
TVSDVPTVSDVPTVSDVPGS

SEQ ID NO: 36 - protein, 8 aa, Artificial Sequence
  Linker
TGLDSPGS

SEQ ID NO: 37 - protein, 14 aa, Artificial Sequence
  Linker
TGLDSPTGLDSPGS

SEQ ID NO: 38 - protein, 20 aa, Artificial Sequence
  Linker
TGLDSPTGLDSPTGLDSPGS

SEQ ID NO: 39 - protein, 86 aa, Artificial Sequence
  Mutated scaffold
EVVAATPTSLLISWRHPHFPTRYYRITYGETGGNSPVQEFTVPLQPPTATISGLKPGVDYTITVYAVTDGRNGRLLSIPISINYRT

SEQ ID NO: 40 - protein, 94 aa, Artificial Sequence
  Mutated scaffold
VSDVPRDLEVVAATPTSLLISWDTHNAYNGYYRITYGETGGNSPVREFTVPHPEVTATISGLKPGVDDTITVYAVTNHHMPLRIFGPISINHRT

SEQ ID NO: 41 - protein, 150 aa, Artificial Sequence
  Mutated scaffold
DGGGIRRSMSGTWYLKAMTVDREFPEMNLESVTPMTLTLLKGHNLEAKVTMLISGRCQEVKAVLGRTKERKKYTADGGKHVAYIIPSAVRDHVIFYSEGQLHGKPVRGVKLVGRDPKNNLEALEDFEKAAGRLSTESILIPRQSETCSPG

SEQ ID NO: 42 - protein, 14 aa, Artificial Sequence
  Linker
TVAAPSTVAAPSGS

SEQ ID NO: 43 - protein, 20 aa, Artificial Sequence
  Linker
TVAAPSTVAAPSTVAAPSGS

SEQ ID NO: 44 - protein, 8 aa, Artificial Sequence
  Linker
GSTVAAPS